(19) United States
(12) Patent Application Publication
(10) Pub. No.: US 2023/0360805 A1
(43) Pub. Date: Nov. 9, 2023

(54) INFORMATION PROCESSING APPARATUS, INFORMATION PROCESSING METHOD, AND STORAGE MEDIUM

(71) Applicant: NEC Corporation, Minato-ku, Tokyo (JP)

(72) Inventors: Takumi OTANI, Tokyo (JP); Takeshi Sasamoto, Tokyo (JP); Junich Inoue, Tokyo (JP)

(73) Assignee: NEC Corporation, Minato-ku, Tokyo (JP)

(21) Appl. No.: 18/029,347

(22) PCT Filed: Oct. 9, 2020
(86) PCT No.: PCT/JP2020/038388

§ 371 Date: Mar. 29, 2023

(51) Int. Cl. G16H 50/80; G16H 10/60; A61B 5/01

(52) U.S. Cl. CPC G16H 50/80; G16H 10/60; A61B 5/01

(57) ABSTRACT

An information processing apparatus according to the present invention includes: an acquisition unit that acquires health information on users who boarded an airplane and seat information on the airplane; a first identifying unit that identifies a first person out of the users based on the health information, the first person having contracted an infectious disease; and a second identifying unit that identifies a second person out of the users based on the seat information, the second person being suspected of having the infectious disease infected from the first person in the airplane.

### TECHNICAL FIELD

The present invention relates to an information processing apparatus, an information processing method, and a storage medium.

### BACKGROUND ART

Patent Literature 1 discloses a body temperature monitoring system for passengers in an airplane that detects a passenger having a fever in the airplane by using infrared cameras mounted behind seats in the airplane to measure body temperatures of the passengers.

### CITATION LIST

### Patent Literature

PTL 1: Japanese Patent Application Laid-Open No. 2017-29219

### SUMMARY OF INVENTION

### Technical Problem

According to the system disclosed in Patent Literature 1, it is possible to detect a user (passenger) having a fever in an airplane and thereby alert and encourage a flight attendant to isolate the user from other users.

However, the user who has contracted an infectious disease does not always have a fever in an airplane. For example, there may be a case where a user's fever is temporarily suppressed in the airplane or a case where a user has contracted an infectious disease in an airport or an airplane but has not yet developed the disease. Thus, even a person who has contracted an infectious disease may be allowed to enter a second country.

Accordingly, the present invention has been made in view of such circumstances and intends to provide an information processing apparatus, an information processing method, and a storage medium that can efficiently detect a person suspected of having contracted an infectious disease.

### Solution to Problem

According to one aspect of the present invention, provided is an information processing apparatus including: an acquisition unit that acquires health information on users who boarded an airplane and seat information on the airplane; a first identifying unit that identifies a first person out of the users based on the health information, the first person having contracted an infectious disease; and a second identifying unit that identifies a second person out of the users based on the seat information, the second person being suspected of having the infectious disease infected from the first person in the airplane.

According to another aspect of the present invention, provided is an information processing method including: acquiring health information on users who boarded an airplane and seat information on the airplane; identifying a first person out of the users based on the health information, the first person having contracted an infectious disease; and identifying a second person out of the users based on the seat information, the second person being suspected of having the infectious disease infected from the first person in the airplane.

According to yet another aspect of the present invention, provided is a storage medium storing a program that causes a computer to perform: acquiring health information on users who boarded an airplane and seat information on the airplane; identifying a first person out of the users based on the health information, the first person having contracted an infectious disease; and identifying a second person out of the users based on the seat information, the second person being suspected of having the infectious disease infected from the first person in the airplane.

### Advantageous Effects of Invention

According to the present invention, an information processing apparatus, an information processing method, and a storage medium that can efficiently detect a person suspected of having contracted an infectious disease can be provided.

### BRIEF DESCRIPTION OF DRAWINGS

FIG. 1 is a schematic diagram illustrating an example of an overall configuration of an information processing system according to a first example embodiment.

FIG. 2 is a diagram illustrating an example of information stored in a token ID information DB according to the first example embodiment.

FIG. 3 is a diagram illustrating an example of information stored in a passage history information DB according to the first example embodiment.

FIG. 4 is a diagram illustrating an example of information stored in an operation information DB according to the first example embodiment.

FIG. 5 is a diagram illustrating an example of information stored in a person-with-fever information DB according to the first example embodiment.

FIG. 6 is a diagram illustrating an example of information stored in a contact person definition information DB according to the first example embodiment.

FIG. 7A is a diagram illustrating an example of a seat map in an airplane according to the first example embodiment.

FIG. 7B is a diagram illustrating an example of a seat map in the airplane according to the first example embodiment.

FIG. 7C is a diagram illustrating an example of a seat map in the airplane according to the first example embodiment.

FIG. 8 is a block diagram illustrating an example of a hardware configuration of a management server according to the first example embodiment.

FIG. 9 is a block diagram illustrating an example of a hardware configuration of a check-in terminal according to the first example embodiment.

FIG. 10 is a block diagram illustrating an example of a hardware configuration of a boarding gate apparatus according to the first example embodiment.

FIG. 11 is a block diagram illustrating an example of a hardware configuration of a signage terminal according to the first example embodiment.

FIG. 12 is a sequence chart illustrating an example of a process in a check-in procedure performed by the information processing system according to the first example embodiment.

FIG. 13 is a sequence chart illustrating an example of a process in an identity verification procedure before boarding performed by the information processing system according to the first example embodiment.

FIG. 14 is a diagram illustrating a state where a face image and a thermography image are captured in a boarding gate terminal according to the first example embodiment.

FIG. 15 is a sequence chart illustrating an example of a data coordination process performed by the management server according to the first example embodiment.

FIG. 16 is a sequence chart illustrating an example of a guidance process performed by the information processing system according to the first example embodiment.

FIG. 17 is a diagram illustrating an example of a guidance window displayed on the signage terminal according to the first example embodiment.

FIG. 18 is a flow chart illustrating an example of a determination process for an inspection target in the management server according to the first example embodiment.

FIG. 19 is a schematic diagram illustrating an example of an overall configuration of an information processing system according to a second example embodiment.

FIG. 20 is a block diagram illustrating an example of a hardware configuration of an automated gate apparatus according to the second example embodiment.

FIG. 21 is a schematic diagram illustrating an example of an external view of an entry gate terminal and an exit gate terminal forming an automatic customs gate apparatus according to the second example embodiment.

FIG. 22A is a block diagram illustrating an example of a hardware configuration of the entry gate terminal of the automatic customs gate apparatus according to the second example embodiment.

FIG. 22B is a block diagram illustrating an example of a hardware configuration of the exit gate terminal of the automatic customs gate apparatus according to the second example embodiment.

FIG. 23 is a sequence chart illustrating an example of a guidance process performed by the information processing system according to the second example embodiment.

FIG. 24 is a flow chart illustrating an example of a determination process for an inspection target in the management server according to the second example embodiment.

FIG. 25 is a sequence chart illustrating an example of a process in an entry inspection procedure performed by the information processing system according to the second example embodiment.

FIG. 26 is a diagram illustrating an example of a guidance window displayed on an automated gate apparatus according to the second example embodiment.

FIG. 27 is a sequence chart illustrating an example of a process in a customs inspection procedure performed by the information processing system according to the second example embodiment.

FIG. 28 is a diagram illustrating an example of a guidance window displayed on the exit gate terminal of the automated customs gate apparatus according to the second example embodiment.

FIG. 29 is a schematic diagram illustrating an example of an overall configuration of an information processing system according to a third example embodiment.

FIG. 30 is a flowchart illustrating an example of a process of identifying a person with a fever in a management server according to the third example embodiment.

FIG. 31 is a diagram illustrating an example of measurement history information on body surface temperatures according to the third example embodiment.

FIG. 32 is a function block diagram of an information processing apparatus according to a fourth example embodiment.

### DESCRIPTION OF EMBODIMENTS

Exemplary example embodiments of the present invention will be described below with reference to the drawings. Throughout the drawings, similar elements or corresponding elements are labeled with the same references, and the description thereof may be omitted or simplified.

### First Example Embodiment

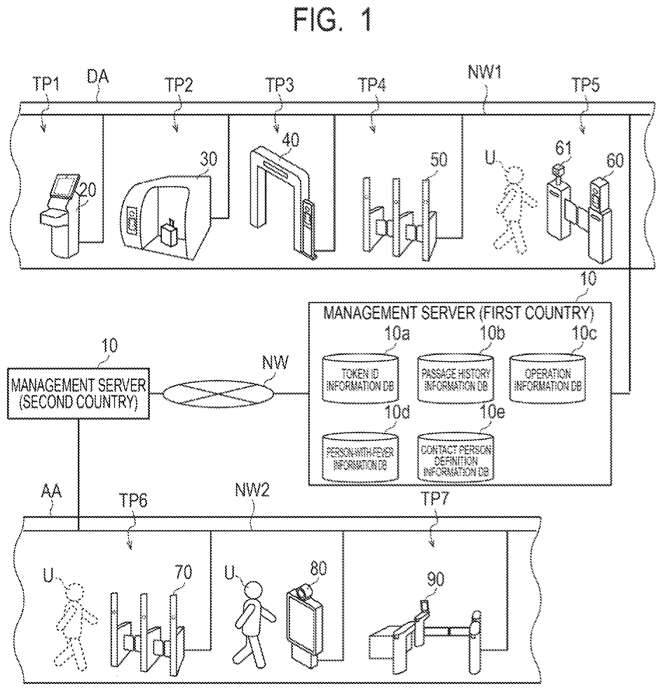

FIG. 1 is a schematic diagram illustrating an example of the overall configuration of an information processing system according to the present example embodiment. The information processing system according to the present example embodiment is a computer system that supports a series of procedures performed on a user U in a first country and a second country, respectively, when the user U departs from the first country at an airport DA of the first country and enters the second country at an airport AA of the second country by an airplane. The information processing system is run by a public institution such as an immigration control bureau or a trustee entrusted with the operation from such an institution, for example.

As illustrated in FIG. 1, the information processing system includes management servers 10, a check-in terminal 20, an automatic baggage drop-off machine 30, a security inspection apparatus 40, an automated gate apparatus 50, a boarding gate apparatus 60, an automated gate apparatus 70, a signage terminal 80, and an automatic customs gate apparatus 90. The management server 10 is connected to other apparatuses via networks NW, NW1, NW2, respectively. The networks NW, NW1, NW2 are each formed of a Wide Area Network (WAN) such as the Internet or a Local Area Network (LAN). The connection scheme may be a wireless scheme without being limited to a wired scheme.

The management servers 10 each are an information processing apparatus that manages various procedures on the user U during entry to or departure from countries. The management server 10 is installed in a facility of an airport company, an airline company, or the like, for example. The management servers 10 of the present example embodiment are installed in the first country and the second country, respectively. Note that the management server 10 is not necessarily required to be provided on a country basis and may be configured as a server used by a plurality of countries in a shared manner. Further, the management server 10 is not required to be a single server and may be configured as a server group including a plurality of servers.

The management server 10 performs identity verification on the user U by matching a face image captured by the check-in terminal 20, which is a face authentication terminal, with a passport face image read from a passport by the check-in terminal 20.

Furthermore, the management server 10 performs identity verification on the user U by matching a face image captured by another face authentication terminal (each of the automatic baggage drop-off machine 30, the security inspection apparatus 40, the automated gate apparatus 50, the boarding gate apparatus 60, or the like) with a registered face image registered in a database, respectively.

Further, as illustrated in FIG. 1, the management server 10 includes a token ID information DB 10a, a passage history information DB 10b, an operation information DB 10c, a person-with-fever information DB 10d, and a contact person definition information DB 10e. These databases are examples, and the management server 10 may further include other databases. Further, a plurality of databases may be aggregated into a single database.

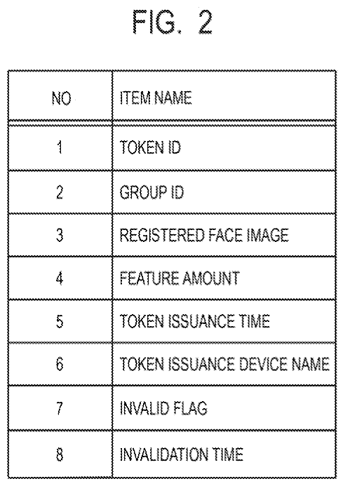

FIG. 2 is a diagram illustrating an example of information stored in the token ID information DB 10a. The token ID information DB 10a has data items of a token ID, a group ID, a registered face image, a feature amount, a token issuance time, a token issuance device name, an invalid flag, and an invalidation time.

The token ID is an identifier that uniquely identifies ID information. The token ID of the present example embodiment is issued by the management server 10 provided that a result of a matching process is that the matching is successful where the matching process is to match a captured face image, which is obtained by the user U capturing his/her face by himself/herself by using a face authentication terminal such as the check-in terminal 20, with a passport face image of the user U read from a passport by the face authentication terminal. Further, for example, after the user U finishes the travel from the first country to the second country, the token ID is invalidated. That is, a token ID is not an identifier used permanently but a onetime ID having a validity period (lifecycle).

Note that the term “matching is successful” in the present example embodiment means that a matching score indicating a similarity between biometric information on the user U and registered biometric information on a registrant is greater than or equal to a predetermined threshold. In contrast, the term “matching is unsuccessful” means that a matching score is less than the predetermined threshold.

The group ID is an identifier for grouping ID information. The registered face image is a face image registered for the user U. In the present example embodiment, a face image of the user U captured during the initial procedure in the airport DA of the first country or a passport face image read from an IC chip of a passport of the user U by a reading device is used as a registered face image stored in the token ID information DB 10a. The feature amount is a value extracted from biometric information (registered face image).

Further, although the term of biometric information in the present example embodiment means a face image and a feature amount extracted from the face image, the biometric information is not limited to a face image and a face feature amount. That is, biometric authentication may be performed by using an iris image, a fingerprint image, a palmprint image, an auricular image, or the like as biometric information on the user U.

The token issuance time is a time that the management server 10 issued a token ID. The token issuance device name is a device name from which a registered face image which triggered issuance of a token ID is acquired. The invalid flag is flag information indicating whether or not a token ID is currently valid. For example, upon issuance of a token ID, the invalid flag is set to a value indicating a state where the token ID is valid. Further, in response to satisfying a predetermined condition, the invalid flag is updated to a value indicating a state where a token ID is invalid. The invalidation time is a timestamp indicating a time the invalid flag is invalidated.

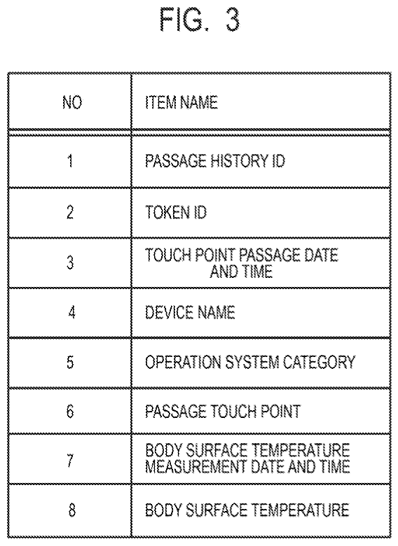

FIG. 3 is a diagram illustrating an example of information stored in the passage history information DB 10b. The passage history information DB 10b has data items of a passage history ID, a token ID, a touch point passage date and time, a device name, an operation system category, a passage touch point, a body surface temperature measurement date and time, and a body surface temperature. The passage history ID is an identifier that uniquely identifies passage history information. The touch point passage date and time is a timestamp indicating a time the user U passes through a touch point. The device name is a machine name of an operation terminal used for a procedure at a touch point. The operation system category is a category of an operation system which an operation terminal belongs to. The passage touch point is a name of a touch point through which the user U passes. The body surface temperature measurement date and time is a timestamp when a body surface temperature of the user U is measured by capturing of a thermography image. The body surface temperature is a temperature measured for a skin surface of the user U.

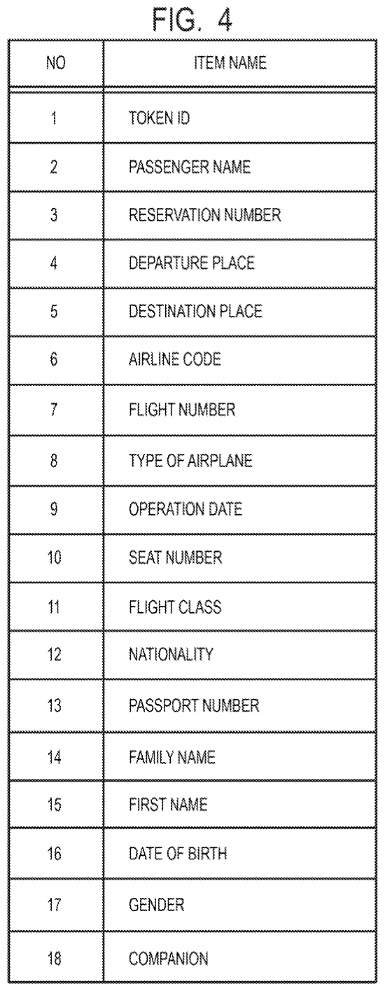

FIG. 4 is a diagram illustrating an example of information stored in the operation information DB 10c. The operation information DB 10c has data items of a token ID, a passenger name, a reservation number, a departure place, a destination place, an airline code, a flight number, a type of airplane, an operation date, a seat number, a flight class, a nationality, a passport number, a family name, a first name, a date of birth, a gender, and a companion. In the present example embodiment, a flight number and a type of airplane are also referred to as flight information.

The reservation number is an identifier that uniquely identifies boarding reservation information.

The airline code is an identifier that uniquely identifies an airline company. The flight class is a class of a seat and may be, for example, first class, business class, economy class, or the like. In general, a seat of a higher flight class has a longer distance to the next seat and a longer distance (seat pitch) to the front and rear seats. Further, services that the user U may receive in an airport and a cabin are also different in accordance with a flight class. The companion is a token ID related to a person accompanied by the user U (for example, an infant, a child, a person requiring support).

Information on a passenger name, a reservation number, a departure place, a destination place, an airline code, a flight number, a type of an airplane, an operation date, a seat number, a nationality, a passport number, a family name, a first name, a date of birth, a gender, or the like may be acquired from a medium such as a passport and a boarding ticket or acquired from a database that manages reservation information (not illustrated) by using passport number, a reservation number, or the like as a key.

In such a way, the operation information DB 10c stores operation information about a predetermined operation in association with a token ID. In the present example embodiment, “predetermined operation” means a procedure operation (check-in/baggage drop-off/security inspection/departure inspection/identity verification on a passenger, or the like) performed at each touch point in an airport.

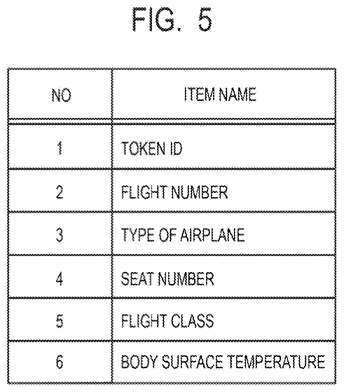

FIG. 5 is a diagram illustrating an example of information stored in the person-with-fever information DB 10d according to the present example embodiment. The person-with-fever information DB 10d has data items of a token ID, a flight number, a type of airplane, a seat number, a flight class, and a body surface temperature. The person-with-fever information DB 10d stores information on a person identified as a person with a fever out of the users U. The term “person with a fever” in the present example embodiment means a person suspected of having contracted an infectious disease.

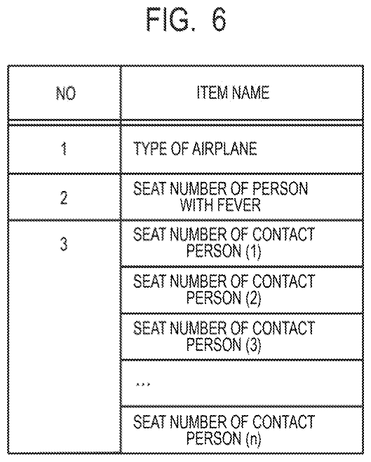

FIG. 6 is a diagram illustrating an example of information stored in the contact person definition information DB 10e according to the example embodiment. The contact person definition information DB 10e has data items of a type of airplane, a seat number of a person with fever, and a seat number of contact person (1) to (n). The term “contact person” in the present example embodiment means a person considered to have an opportunity of contact with a person with a fever in an airplane, in other words, a person suspected of having an infectious disease infected from a person with a fever. That is, the contact person definition information DB 10e defines a seat number of a seat used by a contact person with respect to a seat number of a person with a fever as a reference. Note that the reason why a type of airplane is included as a data item is that the arrangement of seats differs in accordance with a type of an airplane. Further, the seat arrangement may differ for each airline even with the same type of airplanes. In such a case, the contact person definition information DB 10e may further include an airline code in the data items.

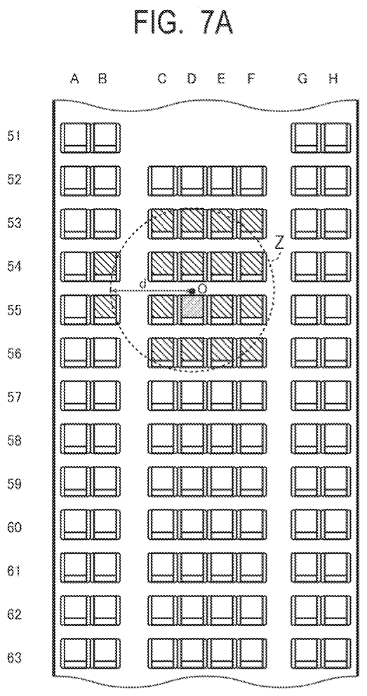

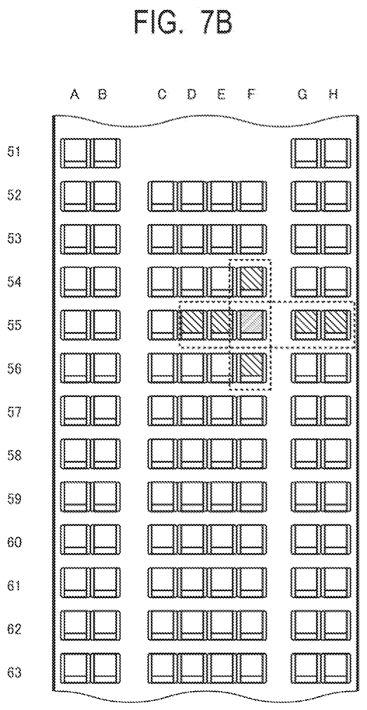

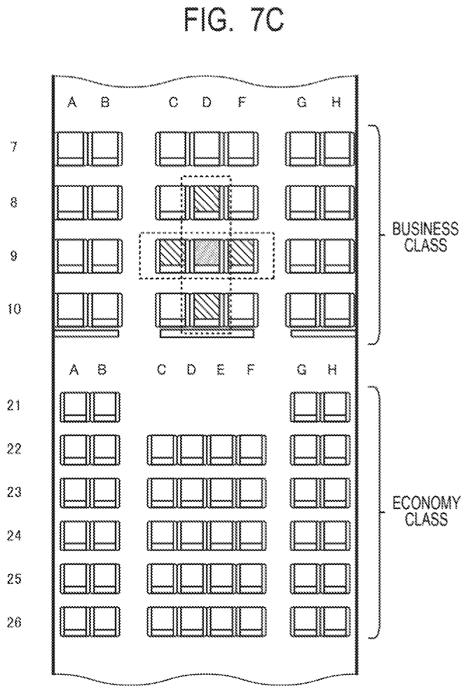

FIG. 7A to FIG. 7C are diagrams illustrating examples of seat maps in an airplane according to the present example embodiment. FIG. 7A indicates a case where the users U using seats within a range of a predetermined distance d from the seat position of a person with a fever are defined as the contact person. When the seat position of a person with a fever is “55D”, seats partially or entirely overlapping the circular region Z having a radius d about the point O are emphasized with hatching.

In details, maximum of 17 users U using the seats (53C, 53D, 53E, 53F, 54B, 54C, 54D, 54E, 54F) in front of a person with a fever, the seats (55B, 55C, 55E, 55F) in the left-right directions of the person with a fever, and the seats (56C, 56D, 56E, 56F) behind the person with a fever are defined as contact persons. Note that a method of defining the positional relationship between a person with a fever and one or more contact persons is not limited to the above. Any definition is possible taking into consideration of a path or a direction when a person with a fever moves, an orientation of the face of the person with a fever when seated, a droplet scattering direction when the person with a fever coughs and sneezes, or the like. For example, the user U seated in front of a person with a fever may be defined as a contact person in preference to the user U seated behind the person with a fever.

Further, as illustrated in FIG. 7B, instead of the distance from a seat of a person with a fever, the users U seated in seats adjacent to the seat of the person with a fever in the front-rear directions and the left-right directions may be uniformly defined as contact persons. FIG. 7B illustrates a case where, with respect to the seat (55F) of a person with a fever as a reference, the users U seated in the seats (54F, 56F) in front of and behind the person with a fever and the seats (55D, 55E, 55G, 55H) up to two seats next to the person with a fever on the left and right are defined as contact persons.

Furthermore, as illustrated in FIG. 7C, the definition range of contact persons may be changed in accordance with a flight class associated with the user U. The business class seats are set to have a longer distance to the next seat and a longer distance (seat pitch) to the front and rear seats than the economy class seats. Thus, FIG. 7C illustrates a case where when the seat of a person with a fever is “9D”, the users U seated in the seats (8D, 10D) in front of and behind the person with a fever and the seats (9C, 9F) next to the person with a fever on the left and right are defined as contact persons.

Next, the apparatuses responsible for procedural operations on the user U in cooperation with the management server 10 in the airport DA and the airport AA according to the present example embodiment will be described.

The check-in terminal 20 is installed in a check-in lobby or a check-in counter in each of the airport DA and the airport AA. Hereafter, the procedural area where the check-in terminal 20 is installed is referred to as “touch point TP1”. The check-in terminal 20 is a self-service terminal operated by the user U by himself/herself to perform a check-in procedure (a boarding procedure). After completion of the check-in procedure at the touch point TP1, the user U moves to a baggage drop-off place or a security inspection site.

The automatic baggage drop-off machine 30 is installed in a region adjacent to a baggage counter (a manned counter) or a region near the check-in terminal 20 in each of the airport DA and the airport AA. Hereafter, the procedural area where the automatic baggage drop-off machine 30 is installed is referred to as “touch point TP2”. The automatic baggage drop-off machine 30 is a self-service terminal operated by the user U by himself/herself to perform a procedure to drop off, to an airline company, baggage not to be carried in the cabin. After completion of the baggage drop-off procedure at the touch point TP2, the user U moves to the security inspection site. When the user U does not drop off his/her baggage, the procedure at the touch point TP2 is omitted.

The security inspection apparatus 40 is installed in the security inspection site (hereafter, referred to as “touch point TP3”) in each of the airport DA and the airport AA. The term “security inspection apparatus” in the present example embodiment is used as a meaning including all of a metal detector that checks whether or not the user U is wearing a metal item that may be a dangerous object, an X-ray inspection device that uses an X-ray to check whether or not a dangerous object is included in carry-on baggage or the like, a passage control device that determines whether or not to permit passage of the user U at an entrance or an exit of the security inspection site, and the like. After completion of the security inspection procedure at the touch point TP3, the user U moves to a departure inspection site.

The automated gate apparatus 50 is installed at the departure inspection site (hereafter, referred to as “touch point TP4”) in each of the airport DA and the airport AA. The automated gate apparatus 50 is an apparatus that automatically performs a departure inspection procedure on the user U. After completion of the departure inspection procedure at the touch point TP4, the user U moves to a departure area where a duty-free shop and a boarding gate are provided.

The boarding gate apparatus 60 is installed to each boarding gate (hereafter, referred to as “touch point TP5”) in each of the airport DA and the airport AA. The boarding gate apparatus 60 is a passage control apparatus that checks whether or not the user U is a passenger of an airplane associated with the boarding gate. After completion of the procedure at the touch point TP5, the user U boards the airplane and departs to the second country. In such a way, the check-in terminal 20, the automatic baggage drop-off machine 30, the security inspection apparatus 40, the automated gate apparatus 50, and the boarding gate apparatus 60 are used when the user U departs from the first country.

The automated gate apparatus 70 is installed at the entry inspection site (hereafter, referred to as “touch point TP6”) in each of the airport DA and the airport AA. The automated gate apparatus 70 is an apparatus that automatically performs an entry inspection procedure on the user U. The hardware configuration of the automated gate apparatus 70 is the same as that of the automated gate apparatus 50 of the airport DA. In the present example embodiment, after completion of the entry inspection procedure at the touch point TP6, the user U moves to a customs inspection site or a quarantine inspection site.

The signage terminal 80 is installed in any place of each of the airport DA and the airport AA. The signage terminal 80 is a display terminal for presenting, to the user U, various guidance information received from the management server 10. The signage terminal 80 of the present example embodiment is at least installed near the exit of the entry inspection site.

The automatic customs gate apparatus 90 is installed in each customs inspection site (hereafter, referred to as “touch point TP7”) in each of the airport DA and the airport AA. The automatic customs gate apparatus 90 is an electronic gate that restricts passage of the user U based on a result of face matching or the like. The user U who is permitted to pass the gate is able to exit the customs inspection site and enter the second country. The user U who is not permitted to pass the gate will be subjected to a separate examination such as being subjected to face-to-face customs inspection with staff in a manned booth (face-to-face lane), for example.

Next, a hardware configuration of devices forming the information processing system will be described. Note that, throughout a plurality of drawings, devices having the same name and differing only in the reference are devices having substantially the same function, and thus, the detailed description thereof will be omitted in the subsequent drawings.

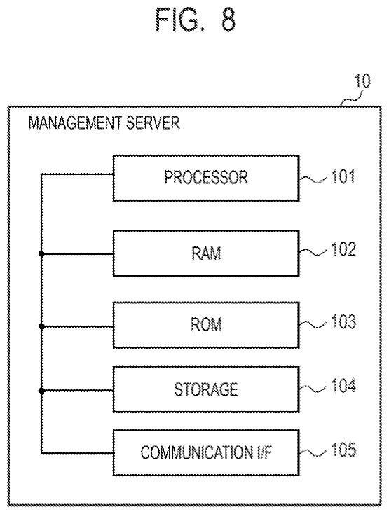

FIG. 8 is a block diagram illustrating an example of a hardware configuration of the management server 10. The management server 10 includes a processor 101, a random access memory (RAM) 102, a read only memory (ROM) 103, a storage 104, and a communication interface (I/F) 105, as a computer that performs calculation, control, and storage. These devices are connected to each other via a bus, a wiring, a drive device, or the like.

The processor 101 has functions of performing predetermined calculation in accordance with a program stored in the ROM 103, the storage 104, or the like and controlling each unit of the management server 10. Further, as the processor 101, one of a central processing unit (CPU), a graphics processing unit (GPU), a field programmable gate array (FPGA), a digital signal processor (DSP), and an application specific integrated circuit (ASIC) may be used, or a plurality thereof may be used in parallel.

The RAM 102 is formed of a volatile storage medium and provides a temporary memory area required for the operation of the processor 101. The ROM 103 is formed of a nonvolatile storage medium and stores information required such as a program used for the operation of the management server 10.

The storage 104 is formed of a nonvolatile storage medium and performs storage of a database, storage of an operating program of the management server 10, or the like. The storage 104 is formed of a hard disk drive (HDD) or a solid state drive (SSD), for example.

The communication I/F 105 is a communication interface based on a specification such as Ethernet (registered trademark), Wi-Fi (registered trademark), 4G, or the like and is a module for communicating with other devices.

The processor 101 loads a program stored in the ROM 103, the storage 104, or the like into the RAM 102 and executes the program to perform a predetermined calculation process. Further, the processor 101 controls each unit of the management server 10, such as the communication I/F 105, based on the program.

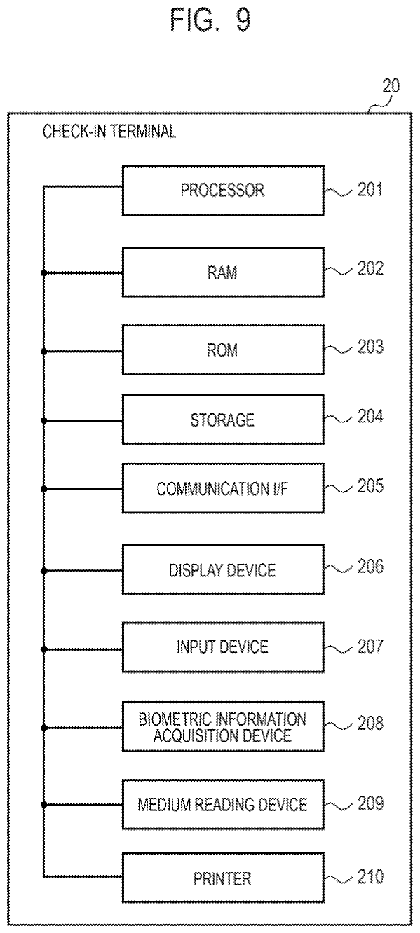

FIG. 9 is a block diagram illustrating an example of the hardware configuration of the check-in terminal 20. The check-in terminal 20 includes a processor 201, a RAM 202, a ROM 203, a storage 204, a communication I/F 205, a display device 206, an input device 207, a biometric information acquisition device 208, a medium reading device 209, and a printer 210. These devices are connected to each other via a bus, a wiring, a drive device, or the like.

The display device 206 is a liquid crystal display, an organic light emitting diode (OLED) display, or the like configured to display a moving image, a static image, a text, or the like and is used for presenting information to the user U.

The input device 207 is a keyboard, a pointing device, a button, or the like and accepts a user operation. The display device 206 and the input device 207 may be formed integrally as a touch panel.

The biometric information acquisition device 208 is a device that acquires a face image of the user U as biometric information on the user U. The biometric information acquisition device 208 is a digital camera having a Complementary Metal-Oxide-Semiconductor (CMOS) image sensor, a Charge Coupled Device (CCD) image sensor, or the like as a light receiving element, for example. The biometric information acquisition device 208 captures an image of a face of the user U standing in front of the device to acquire the face image, for example.

The medium reading device 209 is a device that reads information recorded or stored in a medium carried by the user U. The medium reading device 209 may be, for example, a code reader, an image scanner, a contactless integrated circuit (IC) reader, an optical character reader (OCR) device, or the like. Further, a recording medium or a storage medium may be, for example, a paper airline ticket, a mobile terminal displaying a receipt of an e-ticket, or the like. The printer 210 prints a boarding ticket in which boarding information and guidance information about procedures up to boarding are printed at the time of completion of a check-in procedure.

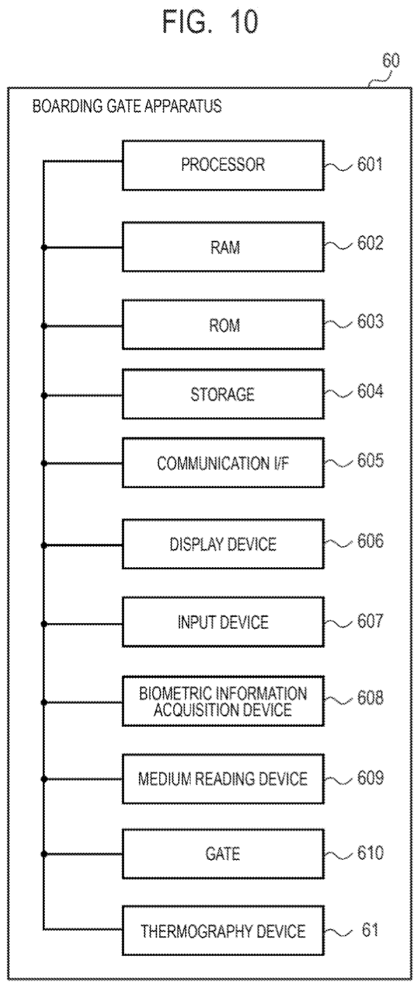

FIG. 10 is a block diagram illustrating an example of the hardware configuration of the boarding gate apparatus 60. The boarding gate apparatus 60 includes a processor 601, a RAM 602, a ROM 603, a storage 604, a communication I/F 605, a display device 606, an input device 607, a biometric information acquisition device 608, a medium reading device 609, a gate 610, and a thermography device 61. These devices are connected to each other via a bus, a wiring, a drive device, or the like.

The gate 610 transitions from a closed state to block passage of the user U during standby to an open state to permit passage of the user U under the control of the processor 601 when identity verification of the user U at the boarding gate apparatus 60 is successful. The type of the gate 610 is not particularly limited and may be, for example, a flapper gate in which one or more flappers provided to one side or both sides of a passage are opened and closed, a turn style gate in which three bars are revolved, or the like.

The thermography device 61 is an image capturing device that analyzes infrared rays emitted from an object and generates a thermography image representing a heat distribution. The thermography device 61 has the following advantages.
(A) It is possible to measure a body surface temperature in a contactless manner with an object to be measured.
(B) It is possible to visualize, as an image, a temperature distribution of a wide area as a plane rather than a temperature value at a point on an object.
(C) It is possible to measure a body surface temperature in real time because the response speed is high.

Because of such advantages, the thermography device 61 according to the present example embodiment is used for measuring the body surface temperature of the user U in the airport DA.

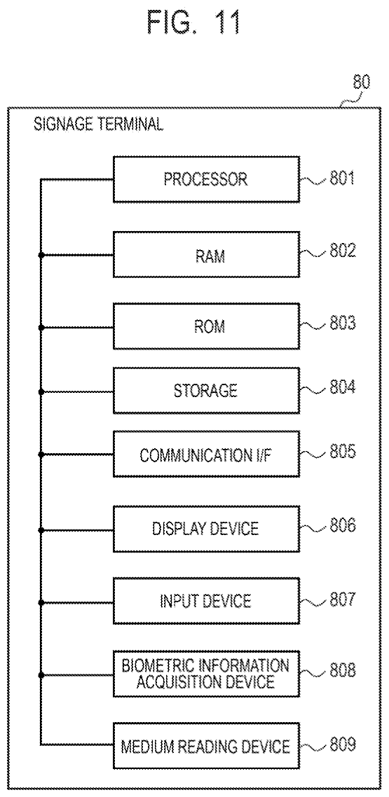

FIG. 11 is a block diagram illustrating an example of the hardware configuration of the signage terminal 80. The signage terminal 80 includes a processor 801, a RAM 802, a ROM 803, a storage 804, a communication I/F 805, a display device 806, an input device 807, a biometric information acquisition device 808, and a medium reading device 809. These devices are connected to each other via a bus, a wiring, a drive device, or the like.

Note that the hardware configurations illustrated in FIG. 8 to FIG. 11 are examples, a device other than the above may be added, and some of the devices may be omitted. Further, some of the devices may be replaced with another device having the same function. Further, some of the functions of the present example embodiment may be provided by another device via a network, or the functions of the present example embodiment may be distributed to and implemented by a plurality of devices. In such a way, the hardware configurations illustrated in FIG. 8 to FIG. 11 can be changed as appropriate.

Next, the operation of the apparatuses in the information processing system according to the present example embodiment will be described with reference to the drawings.

### [Check-In Procedure]

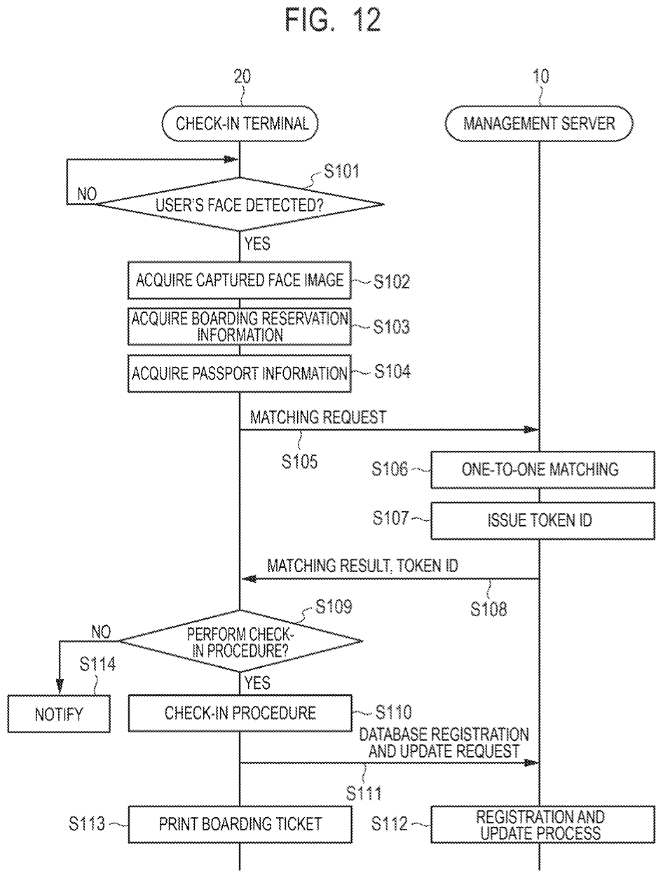

FIG. 12 is a sequence chart illustrating an example of the process in a check-in procedure of the information processing system according to the present example embodiment.

First, the check-in terminal 20 captures an image of the area in front thereof constantly or periodically and determines whether or not a face of a user U standing in front of the check-in terminal 20 is detected in the captured image (step S101). The check-in terminal 20 stands by until a face of a user U is detected in the image by the biometric information acquisition device 208 (step S101: NO).

If the check-in terminal 20 determines that a face of a user U is detected by the biometric information acquisition device 208 (step S101: YES), the check-in terminal 20 captures an image of the face of the user U and acquires the captured face image of the user U as a target face image (step S102). Note that it is preferable to display a window for obtaining user U consent before capturing a face image.

Next, in response to an airline ticket medium being held over the reading area of the medium reading device 209, the check-in terminal 20 acquires boarding reservation information on the user U from the airline ticket medium held over (step S103). The boarding reservation information includes attribute information on the user U (a family name, a first name, a gender, or the like) or flight information (an airline code, a flight number, a boarding date, a departure place, a transit point, a destination place, a seat number, a departure time, an arrival time, or the like).

Next, when a passport is held over the reading area of the medium reading device 209, the check-in terminal 20 acquires passport information on the user U from the passport held over (step S104). The passport information includes a passport face image of the user U, identity verification information, a passport number, information on a country that has issued the passport, or the like.

Next, the check-in terminal 20 requests the management server 10 to match face images (step S105). The data of the matching request includes a captured face image captured at the current place and the passport face image read from the passport.

In response to receiving information from the check-in terminal 20, the management server 10 performs one-to-one matching between the captured face image captured by the check-in terminal 20 and the passport face image (step S106).

Next, the management server 10 issues a token ID provided that the matching result in step S106 is that the matching is successful (step S107) and transmits the matching result and the token ID to the check-in terminal 20 (step S108).

Next, based on the matching result received from the management server 10, the check-in terminal 20 determines whether or not a check-in procedure for the user U is ready to be performed (step S109).

In this step, if the check-in terminal 20 determines that a check-in procedure is not ready to be performed (step S109: NO), the check-in terminal 20 notifies the user U of an error message (step S114) and ends the process.

In contrast, if the check-in terminal 20 determines that the matching result at the management server 10 is that the matching is successful and determines that a check-in procedure on the user U is ready to be performed (step S109: YES), the check-in terminal 20 performs a check-in procedure such as confirmation of an itinerary, selection of a seat, or the like based on input information from the user U (step S110). In response to completion of the check-in procedure, the check-in terminal 20 transmits a database registration and update request to the management server 10 (step S111).

Next, in response to receiving the database registration and update request from the check-in terminal 20, the management server 10 performs a registration process and an update process on the passage history information DB 10b and the operation information DB 10c (step S112). Specifically, the passage history information at the touch point TP1 is registered to the passage history information DB 10b in association with the token ID.

The check-in terminal 20 then prints a boarding ticket describing boarding reservation information and guidance information about procedures up to boarding (step S113) and ends the process.

### [Identity Verification Procedure at Boarding Gate]

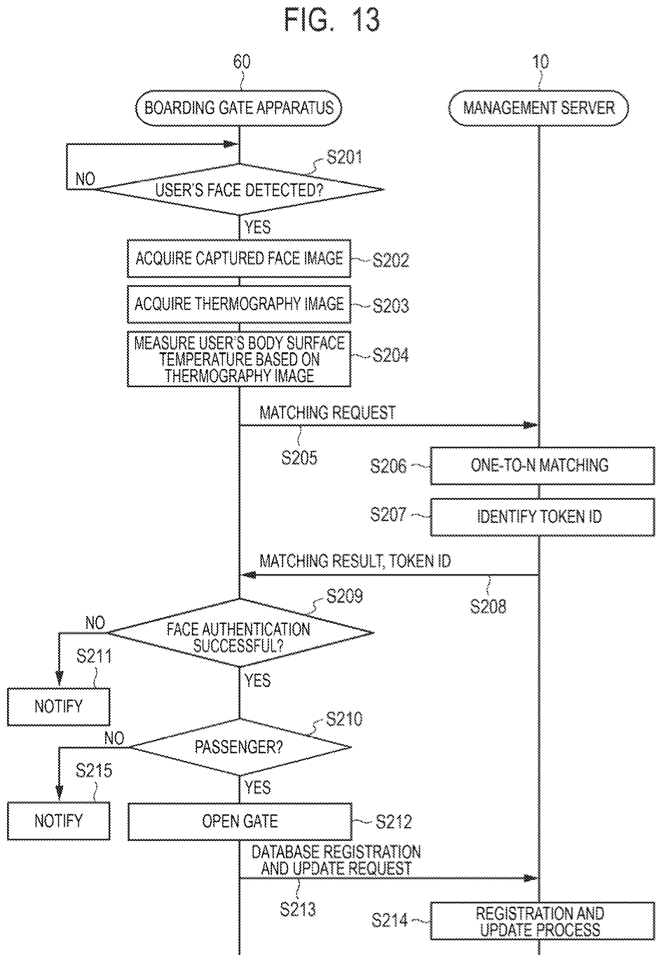

FIG. 13 is a sequence chart illustrating an example of the process in an identity verification procedure at the boarding gate of the information processing system according to the present example embodiment.

First, the boarding gate apparatus 60 captures an image of the area in front of the terminal constantly or periodically and determines whether or not a face of a user U standing in front of the boarding gate apparatus 60 is detected in the captured image (step S201). The boarding gate apparatus 60 stands by until a face of a user U is detected in the image by the biometric information acquisition device 608 (step S201: NO).

If the boarding gate apparatus 60 determines that a face of a user U is detected by the biometric information acquisition device 608 (step S201: YES), the boarding gate apparatus 60 captures an image of the face of the user U and acquires the captured face image of the user U as a target face image (step S202).

Next, the boarding gate apparatus 60 captures an image of the face of the user U by the thermography device 61 and acquires a thermography image (step S203). That is, the boarding gate apparatus 60 captures a thermography image in synchronization with capturing of a captured face image.

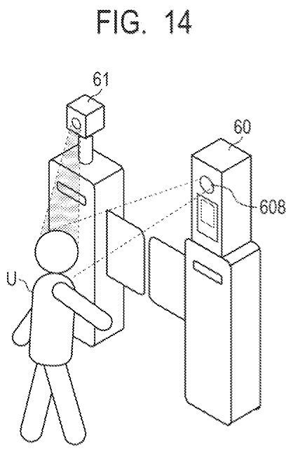

FIG. 14 is a diagram illustrating a state where a face image and a thermography image are captured at the boarding gate apparatus 60. This illustrates an example in which a thermography image including the face of the user U is captured by the thermography device 61 while the face image of the user U is being captured by the biometric information acquisition device 608. It is preferable for the thermography device 61 to start image capturing in response to a timing of capturing performed by the biometric information acquisition device 608 rather than continuously capturing thermography images. This makes it possible to further associate a body surface temperature measured at a boarding gate with a token ID of the user U identified from the face image.

Next, the boarding gate apparatus 60 measures the body surface temperature of the user U based on the thermography image (step S204).

Next, the boarding gate apparatus 60 requests the management server 10 to perform matching of face images (step S205). The data of the matching request includes a captured face image captured at the current place.

In response to receiving data on the matching request from the boarding gate apparatus 60, the management server 10 performs one-to-N matching between the captured face image captured by the boarding gate apparatus 60 and registered face images of registrants stored in the token ID information DB 10a (step S206).

Next, the management server 10 identifies the token ID of the user U provided that the matching result in step S206 is that the matching is successful (step S207).

Next, the management server 10 transmits the matching result and the token ID to the boarding gate apparatus 60 (step S208). Further, to perform a procedure at the boarding gate, the management server 10 transmits operation information (for example, boarding reservation information or passport information) associated with the registered face image to the boarding gate apparatus 60 together with the matching result.

Next, the boarding gate apparatus 60 determines whether or not face authentication of the user U is successful at the management server 10 (step S209).

In this step, if the boarding gate apparatus 60 determines that the matching result at the management server 10 is that the matching is unsuccessful and determines that the face authentication of the user U failed (step S209: NO), the boarding gate apparatus 60 notifies the user U of an error message (step S211) and ends the process.

In contrast, if the boarding gate apparatus 60 determines that the matching result at the management server 10 is that the matching is successful and determines that the face authentication of the user U is successful (step S209: YES), the process proceeds to step S210.

In step S210, the boarding gate apparatus 60 determines whether or not the user U is a passenger of the airplane.

In this step, if the boarding gate apparatus 60 determines that the user U is not a passenger of the airplane (step S210: NO), the boarding gate apparatus 60 notifies the user U of an error message (for example, “Please check the gate number”) (step S215) and ends the process.

In contrast, the boarding gate apparatus 60 determines that the user U is a passenger of the airplane (step S210: YES), the process proceeds to step S212.

In step S212, the boarding gate apparatus 60 opens the gate 610. Accordingly, the user U passes through the boarding gate apparatus 60 and boards the airplane.

Next, in response to the completion of the identity verification procedure of the user U, the boarding gate apparatus 60 transmits a database registration and update request to the management server 10 (step S213).

Then, in response to receiving the database registration and update request from the boarding gate apparatus 60, the management server 10 performs a registration process and an update process on the passage history information DB 10b and the operation information DB 10c (step S214). Specifically, passage history information at the touch point TP5 and measurement history information on the body surface temperature of the user U at the touch point TP5 are registered to the passage history information DB 10b in association with the token ID.

### [Data Coordination Process Between Two Countries]

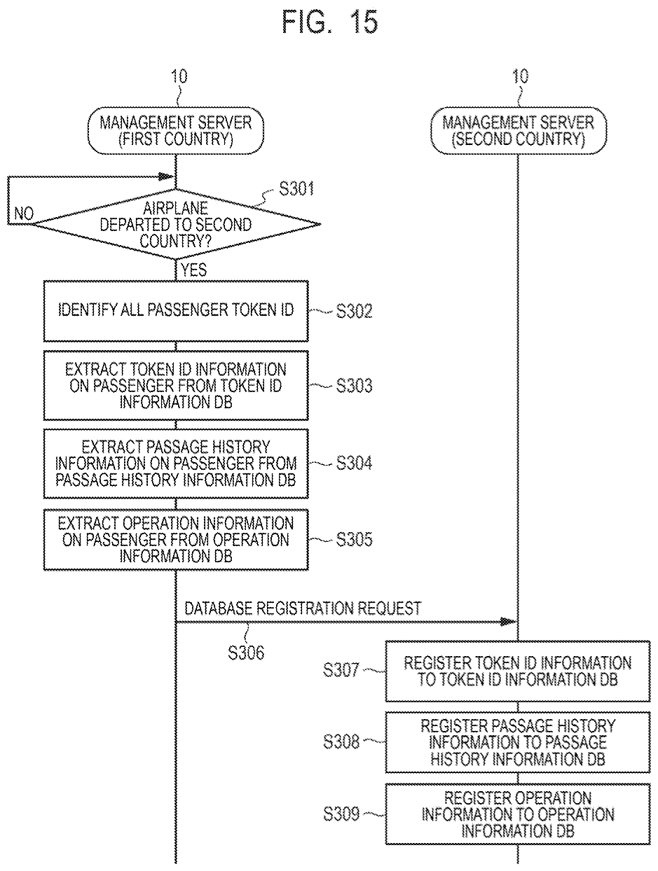

FIG. 15 is a sequence chart illustrating an example of a data coordination process between two countries according to the present example embodiment. This process is performed after an airplane takes off from the airport DA of the first country and before the airplane arrives at the airport AA of the second country, for example.

First, the management server 10 of the first country determines whether or not the airplane has departed to the second country (step S301).

In this step, if the management server 10 of the first country determines that the airplane has departed to the second country (step S301: YES), the process proceeds to step S302.

In contrast, if the management server 10 of the first country determines that the airplane has not yet departed to the second country (step S301: NO), the process of step S301 is repeated.

In step S302, the management server 10 of the first country identifies token IDs from the passage history information DB 10b for all the passengers of the airplane that has departed to the second country.

Next, the management server 10 of the first country extracts token ID information on the passengers from the token ID information DB 10a by using token IDs as keys (step S303).

Next, the management server 10 of the first country extracts passage history information on the passengers from the passage history information DB 10b by using token IDs as keys (step S304).

Next, the management server 10 of the first country extracts operation information on the passengers from the operation information DB 10c by using token IDs as keys (step S305).

Next, the management server 10 of the first country transmits the token ID information, the passage history information, and the operation information extracted for passengers to the management server 10 of the second country and requests for database registration (step S306).

Next, the management server 10 of the second country registers the token ID information received from the management server 10 of the first country to the token ID information DB 10a (step S307).

Next, the management server 10 of the second country registers the passage history information received from the management server 10 of the first country to the passage history information DB 10b (step S308).

The management server 10 of the second country then registers the operation information received from the management server 10 of the first country to the operation information DB 10c (step S309) and ends the process. Accordingly, data related to the passengers are shared between the management server 10 of the first country and the management server 10 of the second country.

[Guidance Process after Entry Inspection]

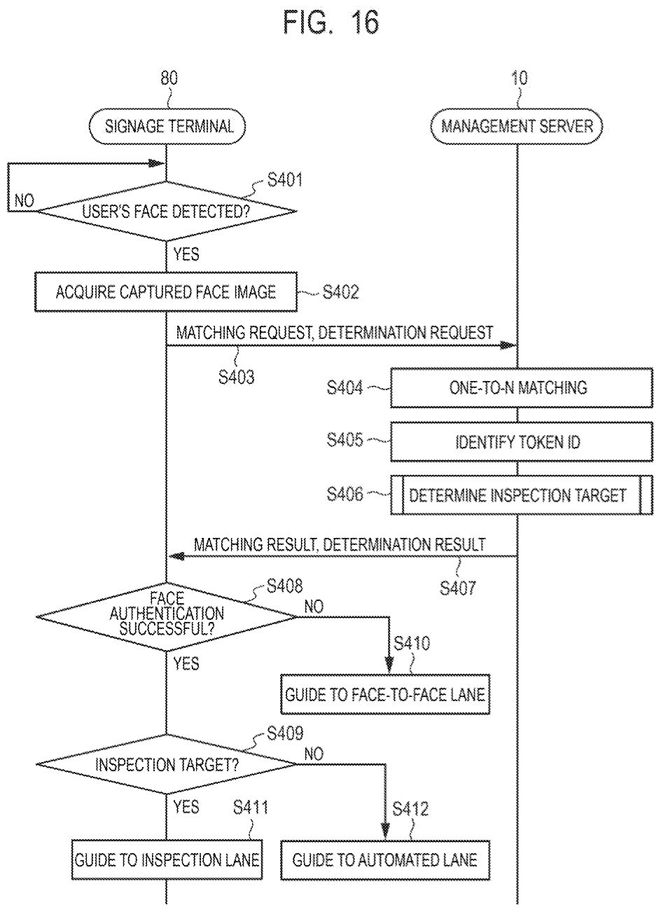

FIG. 16 is a sequence chart illustrating an example of a guidance process of the information processing system according to the present example embodiment.

First, the signage terminal 80 captures an image of the area in front of the terminal constantly or periodically and determines whether or not a face of a user U standing in front of the signage terminal 80 is detected in the captured image (step S401). The signage terminal 80 stands by until a face of a user U is detected in the image by the biometric information acquisition device 808 (step S401: NO).

If the signage terminal 80 determines that a face of a user U is detected by the biometric information acquisition device 808 (step S401: YES), the signage terminal 80 captures an image of a face of the user U and acquires the captured face image of a user U as a target face image (step S402).

Next, the signage terminal 80 requests the management server 10 to perform matching of face images and determination of an inspection target (step S403). The data of the matching request includes a captured face image captured at the current place.

In response to receiving data on the matching request and the determination request from the signage terminal 80, the management server 10 performs one-to-N matching between the captured face image captured by the signage terminal 80 and registered face images of registrants stored in the token ID information DB 10a (step S404).

Next, the management server 10 identifies the token ID of the user U provided that the matching result in step S404 is that the matching is successful (step S405).

Next, the management server 10 determines whether or not the user U is an inspection target based on the identified token ID (step S406). Details of step S406 will be described later.

Next, the management server 10 transmits the matching result and the determination result to the signage terminal 80 (step S407).

Next, based on the matching result received from the management server 10, the signage terminal 80 determines whether or not the face authentication of the user U is successful (step S408).

In this step, if the signage terminal 80 determines that the matching result at the management server 10 is that the matching is unsuccessful and thus determines that the face authentication of the user U is not successful (step S408: NO), the signage terminal 80 displays a guidance message on the display device 806 to guide the user U to a face-to-face lane of the customs inspection site (step S410) and ends the process.

In contrast, if the signage terminal 80 determines that the matching result at the management server 10 is that the matching is successful and thus determines that the face authentication of the user U is successful (step S408: YES), the process proceeds to step S409.

In step S409, based on the determination result received from the management server 10, the signage terminal 80 determines whether or not the user U is an inspection target.

In this step, if the signage terminal 80 determines that the user U is an inspection target (step S409: YES), the signage terminal 80 displays a guidance message on the display device 806 to guide the user U to an inspection lane of the quarantine inspection site (step S411) and ends the process.

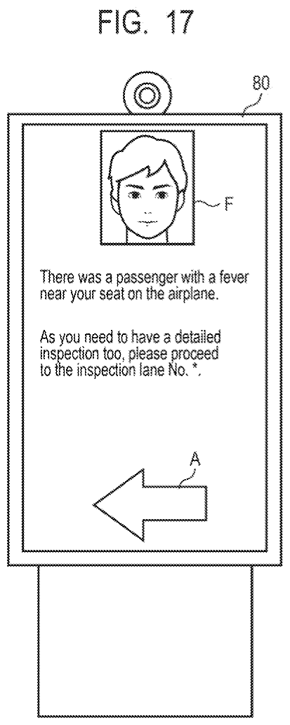

FIG. 17 is a diagram illustrating an example of a guidance window displayed on the signage terminal 80 according to the present example embodiment. In this example, a face image F of the authenticated user U, a message for guiding the user U to an inspection place (“There was a passenger with a fever near your seat on the airplane. As you need to have a detailed inspection too, please proceed to the inspection lane No. *.”), and an arrow A indicating the moving direction are displayed in the window.

In contrast, if the signage terminal 80 determines that the user U is not an inspection target (step S409: NO), the signage terminal 80 displays a guidance message on the display device 806 to guide the user U to an automated lane of the customs inspection site (step S412) and ends the process.

### [Determination Process of Inspection Target]

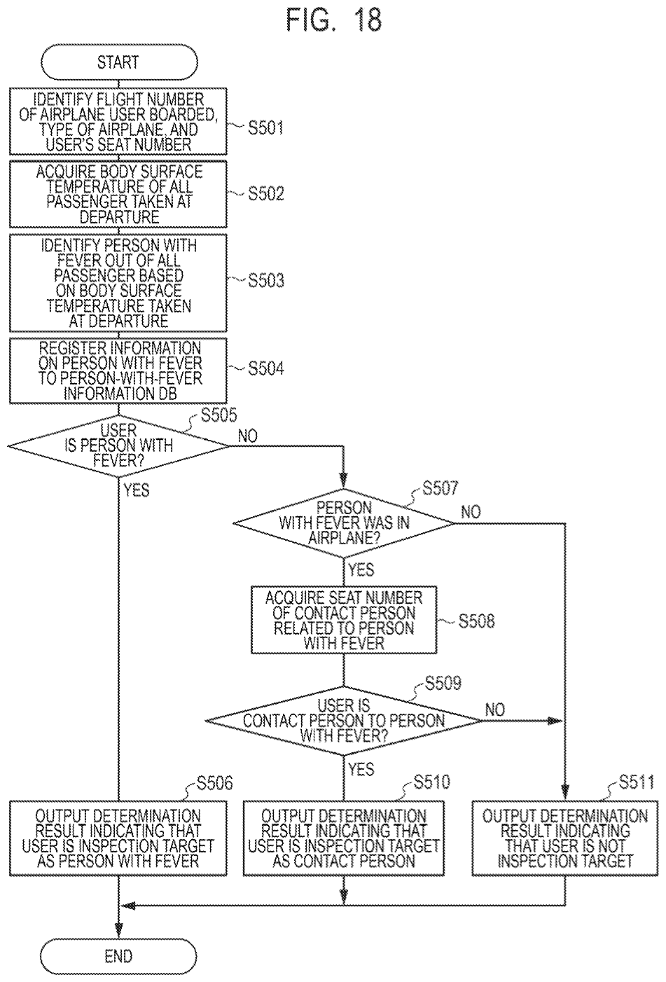

FIG. 18 is a flowchart illustrating an example of a determination process for an inspection target performed by the management server 10 according to the present example embodiment. Although this process illustrates details of step S406 of FIG. 16, the determination method is not limited thereto.

First, the management server 10 references the operation information DB 10c based on the token ID of the user U identified in step S405 of FIG. 16 to identify the flight number and the type of the airplane that the user U has boarded and the seat number of the user U (step S501).

Next, based on the identified flight number, the management server 10 identifies token IDs of all the passengers who have boarded the same airplane from the operation information DB 10c and then acquires body surface temperatures at the departure from the country for all the passengers from the passage history information DB 10b (step S502).

Next, the management server 10 identifies a person with a fever out of all the passengers based on the body surface temperatures at the departure from the country (step S503). In the present example embodiment, when the body surface temperature of a passenger measured at the boarding gate is higher than or equal to a reference value (for example, 37.5 degrees Celsius), the passenger is identified as a person with a fever.

Next, the management server 10 registers person-with-fever information including a token ID of the person with a fever identified out of all the passengers to the person-with-fever information DB 10d (step S504).

Next, the management server 10 determines whether or not the user U is a person with a fever (step S505). In this step, if the management server 10 determines that the user U is a person with a fever (step S505: YES), the process proceeds to step S506.

In contrast, if the management server 10 determines that the user U is not a person with a fever (step S505: NO), the process proceeds to step S507.

In step S506, the management server 10 outputs a determination result indicating that the user U is an inspection target as a person with a fever, and the process ends.

In step S507, the management server 10 references the person-with-fever information DB 10d based on the flight number to determine whether or not a person with a fever was present in the airplane.

In this step, if the management server 10 determines that a person with a fever was present in the airplane (step S507: YES), the process proceeds to step S508.

In contrast, if the management server 10 determines that a person with a fever was not present in the airplane (step S507: NO), the process proceeds to step S511.

In step S508, the management server 10 references the contact person definition information DB 10e based on the seat number of the person with a fever to acquire the seat numbers of contact persons related to the person with a fever.

Next, the management server 10 determines whether or not the user U is a contact person contacted with the person with a fever (step S509). Specifically, it is determined whether or not the seat number of the user U is present in the seat numbers of the contact persons acquired in step S508.

In this step, if the management server 10 determines that the user U is a contact person contacted with the person with a fever (step S509: YES), the process proceeds to step S510.

In contrast, if the management server 10 determines that the user U is not a contact person contacted with the person with a fever (step S509: NO), the process proceeds to step S511.

In step S510, if the management server 10 outputs the determination result indicating that the user U is an inspection target as a contact person, and the process ends.

In step S511, the management server 10 outputs a determination result indicating that the user U is not an inspection target, and the process ends.

As described above, the management server 10 according to the present example embodiment determines whether or not a person with a fever was present in an airplane that the user U has boarded based on user information such as a flight number, a type of an airplane, a seat number, or the like of the airplane associated with the user U identified by face authentication and whether or not the user U is an inspection target as a person with a fever or a contact person. Accordingly, it is possible to efficiently isolate and guide a person suspected of having contracted an infectious disease in the arrival airport.

In particular, the management server 10 according to the present example embodiment can identify, as an inspection target, not only a person with a fever but also a contact person (a so-called close contact person) having an opportunity of contact with a person with a fever at a certain level or higher and therefore achieves an advantageous effect of preventing the spread of infection at an early stage.

### Second Example Embodiment

The information processing system in the present example embodiment will be described below. Note that references common to the references provided in the drawings in the first example embodiment represent the same components. Description of the features common to the first example embodiment will be omitted, and different features will be described in detail.

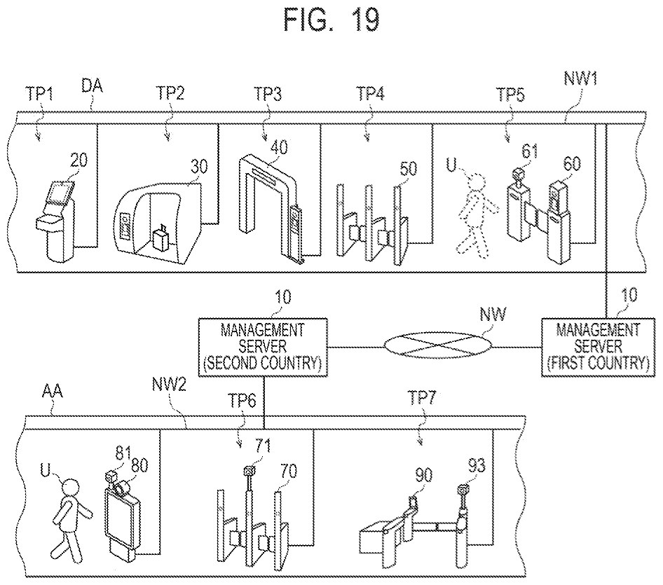

FIG. 19 is a schematic diagram illustrating an example of the overall configuration of the information processing system according to the present example embodiment. As illustrated in FIG. 19, the automated gate apparatus 70, the signage terminal 80, and the automatic customs gate apparatus 90 that are installed in the airport AA have thermography devices 71, 81, and 93, respectively.

In such a way, the present example embodiment differs from the first example embodiment in that the body surface temperature of each user is measured also at the signage terminal 80 installed in the region upstream of the touch point TP6 and the apparatuses at the touch point TP6 and the touch point TP7 in the airport AA of the second country. Accordingly, the management server 10 determines whether or not the user U is an inspection target (a person with a fever or a contact person) based on the measurement history information on body surface temperatures at a plurality of touch points and guides the inspection target to an inspection lane.

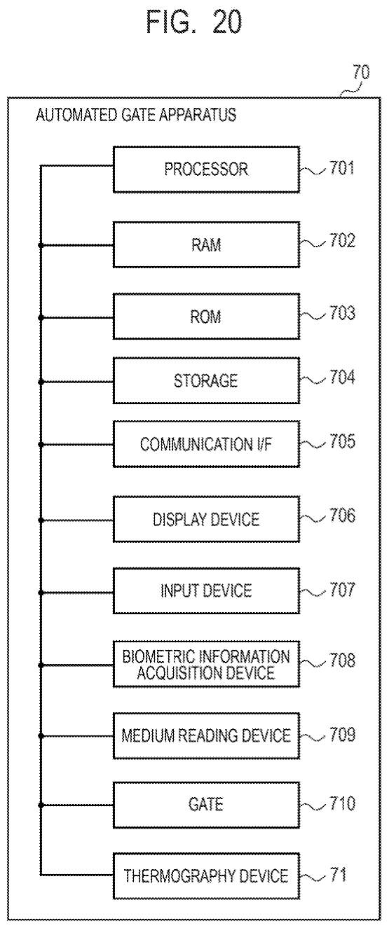

FIG. 20 is a block diagram illustrating an example of a hardware configuration of the automated gate apparatus 70 according to the present example embodiment. The automated gate apparatus 70 includes a processor 701, a RAM 702, a ROM 703, a storage 704, a communication I/F 705, a display device 706, an input device 707, a biometric information acquisition device 708, a medium reading device 709, a gate 710, and a thermography device 71. These devices are connected to each other via a bus, a wiring, a drive device, or the like.

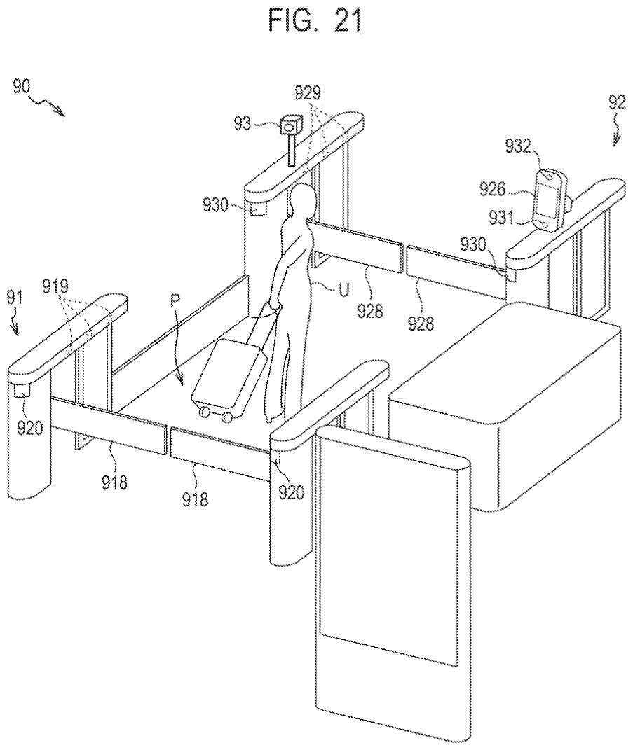

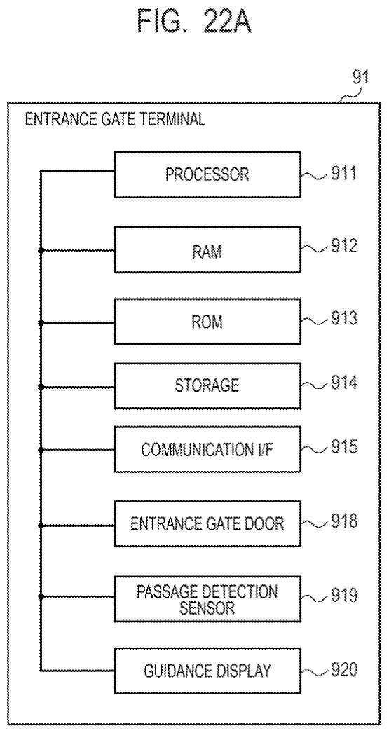

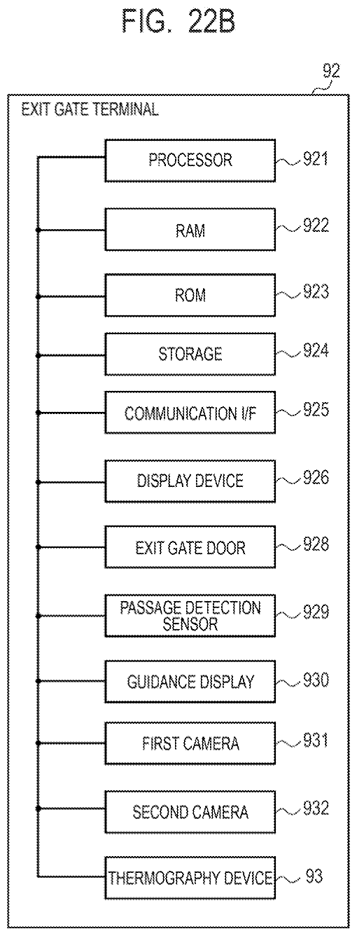

FIG. 21 is a schematic diagram illustrating an external view of an entry gate terminal 91 and an exit gate terminal 92 forming the automatic customs gate apparatus 90. FIG. 22A is a block diagram illustrating an example of a hardware configuration of the entry gate terminal 91. FIG. 22B is a block diagram illustrating an example of a hardware configuration of the exit gate terminal 92.

As illustrated in FIG. 21, the automatic customs gate apparatus 90 includes the entry gate terminal 91 and the exit gate terminal 92. The entry gate terminal 91 and the exit gate terminal 92 are installed on the entry side and on the exit side, respectively, of a gate passage P through which the user U has to pass. In the gate passage P, the user U who has entered the gate passage P is restricted from exiting a space other than the exit gate terminal 92 by a partition plate, a wall, a fence, an inspection table, or the like, for example, installed on both sides along the gate passage P.

As illustrated in FIG. 22A, the entry gate terminal 91 includes a processor 911, a RAM 912, a ROM 913, a storage 914, a communication I/F 915, an entry gate door 918, a passage detection sensor 919, and a guidance display 920. These devices are connected to each other via a bus, a wiring, a drive device, or the like.

The entry gate door 918 is an open/close door that performs a door opening operation and a door closing operation under the control of the processor 911 and transitions between a door opened state that permits passage of the user U and a door closed state that blocks passage of the user U. The opening/closing type of the entry gate door 918 is not particularly limited and may be, for example, a flapper type, a slide type, a revolving type, or the like.

In response to detecting passage of the user U, the passage detection sensor 919 outputs an output signal indicating the passage of the user U. The processor 911 can determine whether or not the user U has passed through the entry gate terminal 91 and entered the gate passage P based on the output signals from a plurality of passage detection sensors 919 and the output order thereof.

Each guidance display 920 displays display indicating whether or not to permit entry to the gate passage P under the control of the processor 911. When the entry gate door 918 is in an open state, the guidance display 920 displays that entry to the gate passage P is permitted. Further, when the entry gate door 918 is in a closed state, the guidance display 920 displays that entry to the gate passage P is not allowed. The guidance display 920 can display whether or not to permit entry to the gate passage P by color display, symbol display, text display, or the like, for example.

As illustrated in FIG. 22B, the exit gate terminal 92 includes a processor 921, a RAM 922, a ROM 923, a storage 924, a communication I/F 925, a display device 926, an exit gate door 928, a passage detection sensor 929, a guidance display 930, a first camera 931, a second camera 932, and a thermography device 93. These devices are connected to each other via a bus, a wiring, a drive device, or the like.

The exit gate door 928 is an open/close door that performs a door opening operation and a door closing operation under the control of the processor 921 and transitions between a door closed state that blocks passage of the user U and a door opened state that permits passage of the user U.

The first camera 931 is a long-range camera that has an image-capturing range including at least the inside of the gate passage P and is able to capture an image of a more distant area than the second camera 932. The second camera 932 is a short-range camera having an image-capturing range including at least the area in front of the exit gate terminal 92. Note that the positions at which the first camera 931 and the second camera 932 are provided are not particularly limited and can be any position where respective image-capturing ranges can be achieved.

### [Guidance Process Before Entry Inspection]

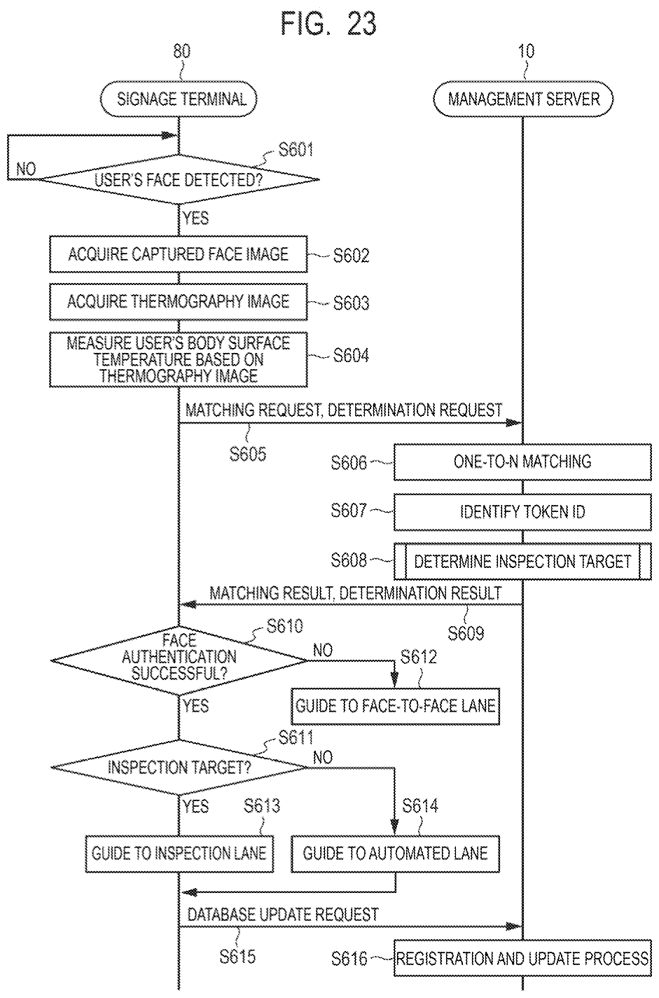

FIG. 23 is a sequence chart illustrating an example of a guidance process of the information processing system according to the present example embodiment.

First, the signage terminal 80 captures an image of the area in front of the terminal constantly or periodically and determines whether or not a face of a user U standing in front of the signage terminal 80 is detected in the captured image (step S601). The signage terminal 80 stands by until a face of a user U is detected in the image by the biometric information acquisition device 808 (step S601: NO).

If the signage terminal 80 determines that a face of a user U is detected by the biometric information acquisition device 808 (step S601: YES), the signage terminal 80 captures an image of the face of the user U and acquires the captured face image of the user U as a target face image (step S602).

Next, the signage terminal 80 captures the face of the user U by the thermography device 81 and acquires a thermography image (step S603). That is, the signage terminal 80 captures a thermography image in synchronization with capturing of the captured face image.

Next, the signage terminal 80 measures the body surface temperature of the user U based on the thermography image (step S604).

Next, the signage terminal 80 requests the management server 10 to perform matching of face images (step S605). The data of the matching request includes a captured face image captured at the current place.

In response to receiving data on the matching request from the signage terminal 80, the management server 10 performs one-to-N matching between the captured face image captured by the signage terminal 80 and registered face images of registrants stored in the token ID information DB 10a (step S606).

Next, the management server 10 identifies the token ID of the user U provided that the matching result in step S606 is that the matching is successful (step S607).

Next, the management server 10 determines whether or not the user U is an inspection target based on the identified token ID (step S608). Details of step S608 will be described later.

Next, the management server 10 transmits the matching result and the determination result to the signage terminal 80 (step S609).

Next, based on the matching result received from the management server 10, the signage terminal 80 determines whether or not the face authentication of the user U is successful (step S610).

In this step, if the signage terminal 80 determines that the matching result at the management server 10 is that the matching is unsuccessful and thus determines that the face authentication of the user U is not successful (step S610: NO), the signage terminal 80 guides the user U to a face-to-face lane of the entry inspection site (step S612) and ends the process.

In contrast, if the signage terminal 80 determines that the matching result at the management server 10 is that the matching is successful and thus determines that the face authentication of the user U is successful (step S610: YES), the process proceeds to step S611.

In step S611, based on the determination result received from the management server 10, the signage terminal 80 determines whether or not the user U is an inspection target.

In this step, if the signage terminal 80 determines that the user U is an inspection target (step S611: YES), the signage terminal 80 displays a guidance message on the display device 806 to guide the user U to an inspection lane of the quarantine inspection site (step S613). The process then proceeds to step S615.

In contrast, if the signage terminal 80 determines that the user U is not an inspection target (step S611: NO), the signage terminal 80 displays a guidance message on the display device 806 to guide the user U to an automated lane of the entry inspection site (step S614). The process then proceeds to step S615.

In step S615, the signage terminal 80 transmits a database registration and update request to the management server 10.

Then, in response to receiving the database registration and update request from the signage terminal 80, the management server 10 performs a registration process and an update process on the passage history information DB 10b and the operation information DB 10c (step S616). Specifically, passage history information at the installation place of the signage terminal 80 and measurement history information on the body surface temperature of the user U at the signage terminal 80 are registered to the passage history information DB 10b in association with the token ID.

### [Determination Process for Inspection Target]

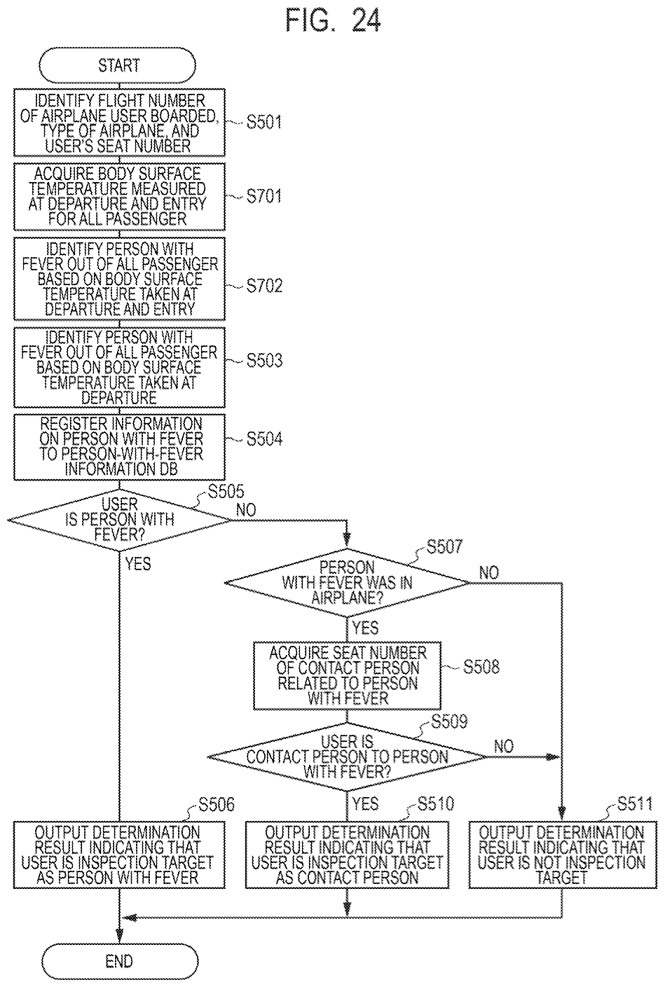

FIG. 24 is a flowchart illustrating an example of a determination process for an inspection target performed by the management server 10 according to the present example embodiment. The process of FIG. 24 differs from that of FIG. 18 described above only in step S701 and step S702. Thus, different features will be described below.

In step S701, the management server 10 acquires body surface temperatures measured at departure from the country and at entry to the country for all the passengers of the airplane that the user U has boarded whose token ID has been identified by face authentication.

In the present example embodiment, the body surface temperature at the entry to the country for each person is measured at the installation place of the signage terminal 80, the entry inspection site (touch point TP6), and the customs inspection site (touch point TP7), respectively. However, the number of data on the body surface temperature at entry to the country acquired from each person differs in accordance with the progress status of procedures in the airport AA.

For example, when a passenger X has completed the customs inspection procedure so far, the number of data on the body surface temperature acquired for the passenger X is three. On the other hand, when another passenger Y has completed the entry inspection procedure so far, the number of data on the body surface temperature acquired for the passenger Y is two.

In step S702, the management server 10 identifies a person with a fever out of all the passengers based on the body surface temperatures of all the passengers measured at departure from the country and at entry to the country. The process then proceeds to step S503.

### [Entry Inspection Procedure]

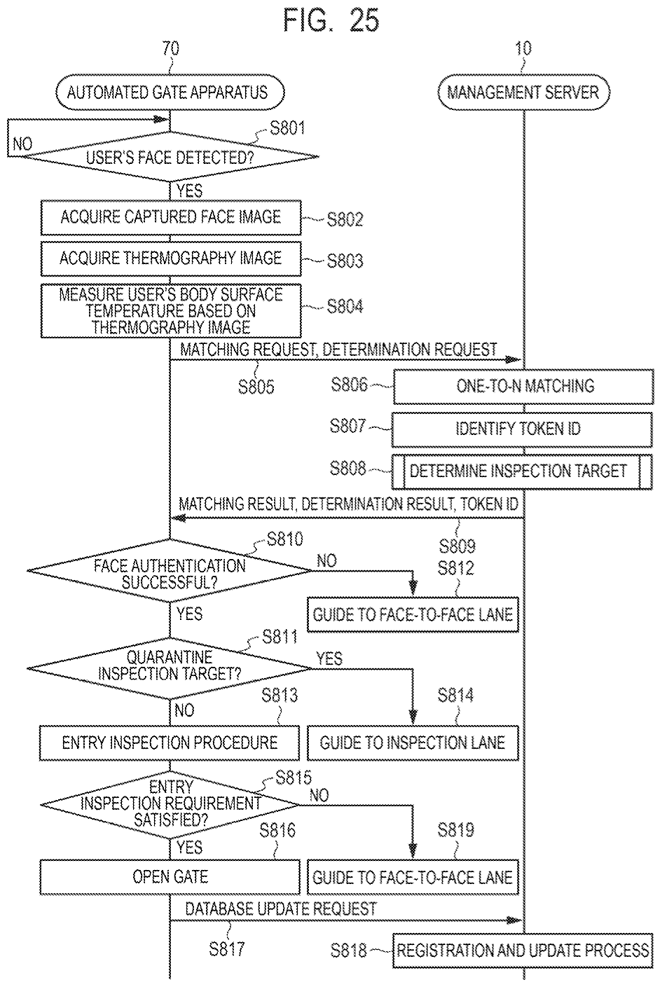

FIG. 25 is a sequence chart illustrating an example of a process in an entry inspection procedure performed by the information processing system according to the present example embodiment.

First, the automated gate apparatus 70 captures an image of the area in front of the terminal constantly or periodically and determines whether or not a face of a user U standing in front of the automated gate apparatus 70 is detected in the captured image (step S801). The automated gate apparatus 70 stands by until a face of a user U is detected in the image by the biometric information acquisition device 708 (step S801: NO).

If the automated gate apparatus 70 determines that a face of a user U is detected by the biometric information acquisition device 708 (step S801: YES), the automated gate apparatus 70 captures an image of the face of the user U and acquires the captured face image of the user U as a target face image (step S802).

Next, the automated gate apparatus 70 captures the face of the user U by the thermography device 71 and acquires a thermography image (step S803). That is, the automated gate apparatus 70 captures a thermography image in synchronization with capturing of the captured face image.

Next, the automated gate apparatus 70 measures the body surface temperature of the user U based on the thermography image (step S804).

Next, the automated gate apparatus 70 requests the management server 10 to perform matching of face images and determination of an inspection target (step S805). The data of the matching request includes a captured face image captured at the current place.

In response to receiving data on the matching request from the automated gate apparatus 70, the management server 10 performs one-to-N matching between the captured face image captured by the automated gate apparatus 70 and registered face images of registrants stored in the token ID information DB 10a (step S806).

Next, the management server 10 identifies the token ID of the user U provided that the matching result in step S806 is that the matching is successful (step S807).

Next, the management server 10 determines whether or not the user U is an inspection target based on the identified token ID (step S808). The process of step S808 is the same as that of FIG. 24 described above.

Next, the management server 10 transmits the matching result and the token ID to the automated gate apparatus 70 (step S809). Further, to perform the entry inspection procedure, the management server 10 transmits operation information (for example, boarding reservation information or passport information) associated with the registered face image to the automated gate apparatus 70 together with the matching result.

Next, based on the matching result received from the management server 10, the automated gate apparatus 70 determines whether or not the face authentication of the user U is successful (step S810).

In this step, if the automated gate apparatus 70 determines that the matching result at the management server 10 is that the matching is unsuccessful and thus determines that the face authentication of the user U is not successful (step S810: NO), the automated gate apparatus 70 displays a guidance message on the display device 706 to guide the user U to a face-to-face lane of the entry inspection site (step S812) and ends the process.

In contrast, if the automated gate apparatus 70 determines that the matching result at the management server 10 is that the matching is successful and thus determines that the face authentication of the user U is successful (step S810: YES), the process proceeds to step S811.

In step S811, based on the determination result received from the management server 10, the automated gate apparatus 70 determines whether or not the user U is a target of quarantine inspection (inspection target).

In this step, if the automated gate apparatus 70 determines that the user U is an inspection target (step S811: YES), the automated gate apparatus 70 displays a guidance message on the display device 706 to guide the user U to an inspection lane of the quarantine inspection site (step S814).

In contrast, if the automated gate apparatus 70 determines that the user U is not an inspection target (step S811: NO), the process then proceeds to step S813.

In step S813, the automated gate apparatus 70 performs the entry inspection procedure. The process then proceeds to step S815.

In step S815, based on the operation information on the user U, the automated gate apparatus 70 determines whether or not the user U is a person who satisfies requirements of entry inspection.

In this step, if the automated gate apparatus 70 determines that the user U is a person who satisfies the requirements of the entry inspection (step S815: YES), the automated gate apparatus 70 opens the gate 510 (step S816). The process then proceeds to step S817.

In contrast, if the automated gate apparatus 70 determines that the user U is not a person who satisfies the requirements of the entry inspection (step S815: NO), the automated gate apparatus 70 displays a guidance message on the display device 706 to guide the user U to a face-to-face lane of the entry inspection site (step S819) and ends the process.

In step S817, in response to the completion of the entry inspection procedure, the automated gate apparatus 70 transmits a database registration and update request to the management server 10.

Then, in response to receiving the database registration and update request from the automated gate apparatus 70, the management server 10 performs a registration process and an update process on the passage history information DB 10b and the operation information DB 10c (step S818). Specifically, passage history information at the touch point TP6 and measurement history information on the body surface temperature of the user U at the touch point TP6 are registered to the passage history information DB 10b in association with the token ID.

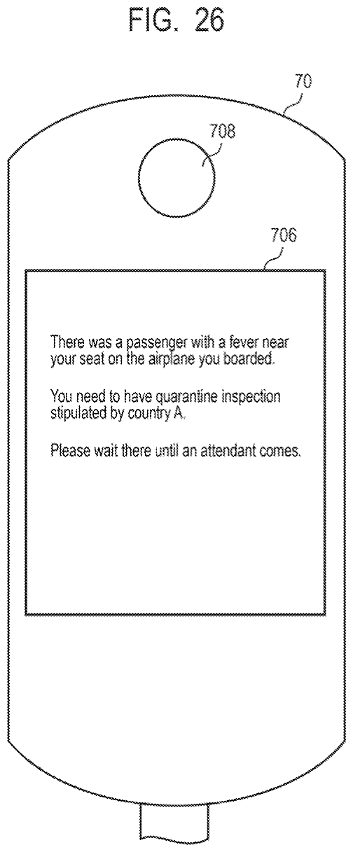

FIG. 26 is a diagram illustrating an example of a guidance window displayed on the automated gate apparatus 70 according to the present example embodiment. In this example, a guidance message about quarantine inspection to the user U (“There was a passenger with a fever near your seat on the airplane you boarded. You need to have quarantine inspection stipulated by country A. Please wait there until an attendant comes.”) is displayed on the display device 706 of the automated gate apparatus 70.

### [Customs Inspection Procedure]

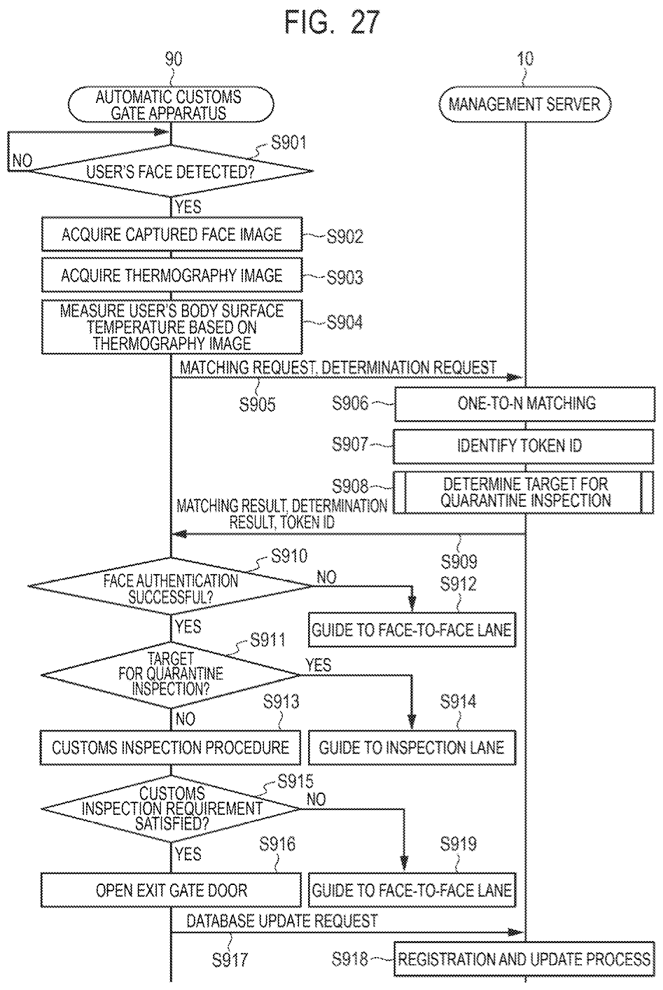

FIG. 27 is a sequence chart illustrating an example of a process in a customs inspection procedure performed by the information processing system according to the present example embodiment.

First, the automatic customs gate apparatus 90 captures an image of the area in front of the terminal constantly or periodically and determines whether or not a face of a user U standing in front of the automatic customs gate apparatus 90 is detected in the captured image (step S901). The automatic customs gate apparatus 90 stands by until a face of a user U is detected in the image by the first camera 931 and the second camera 932 (step S901: NO).

If the automatic customs gate apparatus 90 determines that a face of a user U is detected by the first camera 931 or the second camera 932 (step S901: YES), the automatic customs gate apparatus 90 captures an image of the face of the user U and acquires the captured face image of the user U as a target face image (step S902).

Next, the automatic customs gate apparatus 90 captures the face of the user U by the thermography device 93 and acquires a thermography image (step S903). That is, the automatic customs gate apparatus 90 captures a thermography image in synchronization with capturing of the captured face image.

Next, the automatic customs gate apparatus 90 measures the body surface temperature of the user U based on the thermography image (step S904).

Next, the automatic customs gate apparatus 90 requests the management server 10 to perform matching of face images and determination of a target for quarantine inspection (step S905). The data of the matching request includes a captured face image captured at the current place.

In response to receiving data on the matching request from the automatic customs gate apparatus 90, the management server 10 performs one-to-N matching between the captured face image captured by the automatic customs gate apparatus 90 and registered face images of registrants stored in the token ID information DB 10a (step S906).

Next, the management server 10 identifies the token ID of the user U provided that the matching result in step S906 is that the matching is successful (step S907).

Next, the management server 10 determines whether or not the user U is a target for quarantine inspection based on the identified token ID (step S908). The process of step S908 is the same as that of FIG. 24 described above.

Next, the management server 10 transmits the matching result, the determination result, and the token ID to the automatic customs gate apparatus 90 (step S909). Further, to perform the customs inspection procedure, the management server 10 transmits operation information (for example, boarding reservation information or passport information) associated with the registered face image to the automatic customs gate apparatus 90 together with the matching result.

Next, based on the matching result received from the management server 10, the automatic customs gate apparatus 90 determines whether or not the face authentication of the user U is successful (step S910).

In this step, if the automatic customs gate apparatus 90 determines that the matching result at the management server 10 is that the matching is unsuccessful and thus determines that the face authentication of the user U is not successful (step S910: NO), the automatic customs gate apparatus 90 displays a guidance message on the display device 926 to guide the user U to a face-to-face lane of the customs inspection site (step S912) and ends the process.

In contrast, if the automatic customs gate apparatus 90 determines that the matching result at the management server 10 is that the matching is successful and thus determines that the face authentication of the user U is successful (step S910: YES), the process proceeds to step S911.

In step S911, based on the determination result received from the management server 10, the automatic customs gate apparatus 90 determines whether or not the user U is a target for quarantine inspection.

In this step, if the automatic customs gate apparatus 90 determines that the user U is a target for quarantine inspection (step S911: YES), the automatic customs gate apparatus 90 displays a guidance message on the display device 926 to guide the user U to an inspection lane of the quarantine inspection site (step S914) and ends the process.

In contrast, if the automatic customs gate apparatus 90 determines that the user U is not a target for quarantine inspection (step S911: NO), the process then proceeds to step S913.

In step S913, the automatic customs gate apparatus 90 performs the customs inspection procedure. The process then proceeds to step S915.

In step S915, based on the operation information on the user U, the automatic customs gate apparatus 90 determines whether or not the user U is a person who satisfies requirements of customs inspection.

In this step, if the automatic customs gate apparatus 90 determines that the user U is a person who satisfies the requirements of the customs inspection (step S915: YES), the automatic customs gate apparatus 90 opens the exit gate door 928 (step S916). The process then proceeds to step S917.

In contrast, if the automatic customs gate apparatus 90 determines that the user U is not a person who satisfies the requirements of the customs inspection (step S915: NO), the automatic customs gate apparatus 90 displays a guidance message on the display device 926 to guide the user U to a face-to-face lane of the customs inspection site (step S919) and ends the process.

In step S917, in response to the completion of the customs inspection procedure, the automatic customs gate apparatus 90 transmits a database registration and update request to the management server 10.

Then, in response to receiving the database registration and update request from the automatic customs gate apparatus 90, the management server 10 performs a registration process and an update process on the passage history information DB 10b and the operation information DB 10c (step S918). Specifically, passage history information at the touch point TP7 and measurement history information on the body surface temperature of the user U at the touch point TP7 are registered to the passage history information DB 10b in association with the token ID.

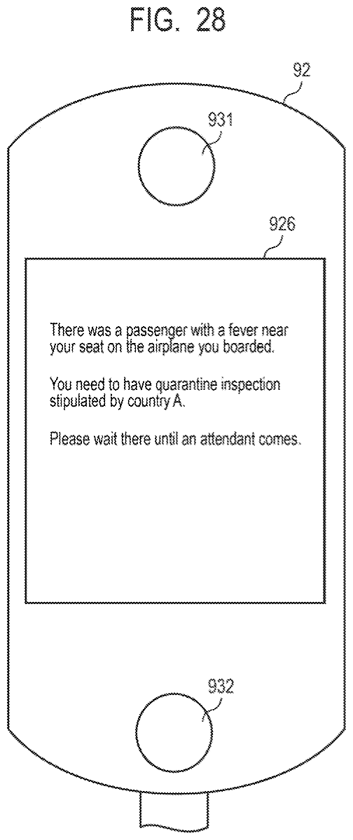

FIG. 28 is a diagram illustrating an example of a guidance window displayed on the exit gate terminal 92 of the automatic customs gate apparatus 90. In this example, a guidance message about quarantine inspection to the user U (“There was a passenger with a fever near your seat on the airplane you boarded. You need to have quarantine inspection stipulated by country A. Please wait there until an attendant comes.”) is displayed on the display device 926 of the exit gate terminal 92.

As described above, the present example embodiment can achieve, in addition to the advantageous effect of the first example embodiment, an advantageous effect of being able to detect a user U who has a fever on board during a flight of an airplane or during a procedure after entry to the country based on body surface temperatures measured at the touch point TP6 and the touch point TP7 in the airport AA of the second country and guide the user U to an inspection lane of the quarantine inspection site.

### Third Example Embodiment

The information processing system in the present example embodiment will be described below. Note that references common to the references provided in the drawings in the first example embodiment represent the same components. Description of the features common to the first example embodiment will be omitted, and different features will be described in detail.

The present example embodiment differs from the first example embodiment in having a function of identifying a person with a fever based on measurement history information about body surface temperatures measured at the touch points TP1 to TP5 of the airport DA and the touch points TP6 to TP7 of the airport AA.

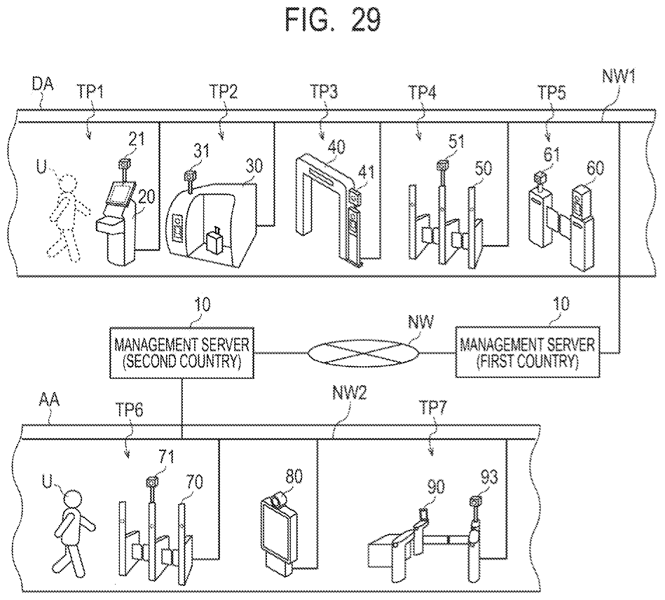

FIG. 29 is a schematic diagram illustrating an example of the overall configuration of the information processing system according to the present example embodiment. As illustrated in FIG. 29, the check-in terminal 20, the automatic baggage drop-off machine 30, the security inspection apparatus 40, the automated gate apparatus 50, the boarding gate apparatus 60, the automated gate apparatus 70, and the automatic customs gate apparatus 90 of the present example embodiment have thermography devices 21, 31, 41, 51, 61, 71, and 93, respectively.

[Process of Identifying Person with Fever]

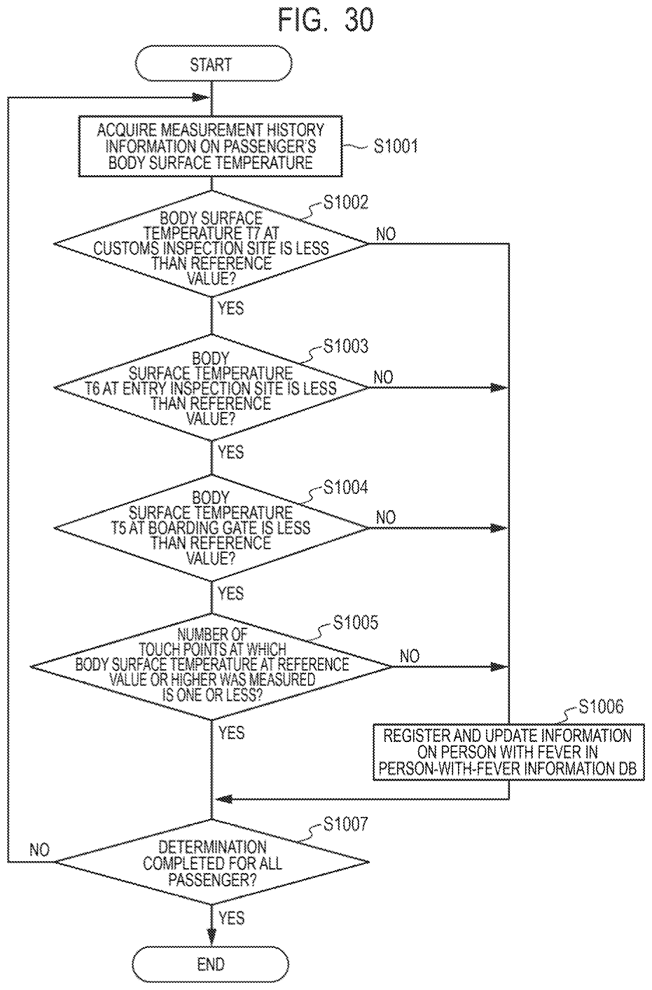

FIG. 30 is a flowchart illustrating an example of a process of identifying a person with a fever performed by the management server 10 according to the present example embodiment. For example, this process may be performed instead of steps S701 and S702 in FIG. 24 described above.

First, the management server 10 identifies token IDs of all the passengers who boarded the same airplane from the operation information DB 10c based on an identified flight number and then references the passage history information DB 10b by using the token ID of each passenger as a key to acquire measurement history information on the body surface temperature of the passengers (step S1001).

Next, the management server 10 determines whether or not a body surface temperature T7 at the customs inspection site (touch point TP7) is lower than a predetermined reference value (step S1002). In the present example embodiment, illustration is provided as the reference value being 37.5 degrees Celsius.

In this step, if the management server 10 determines that the body surface temperature T7 at the customs inspection site is lower than the predetermined reference value (step S1002: YES), the process proceeds to step S1003.

In contrast, if the management server 10 determines that the body surface temperature T7 at the customs inspection site is higher than or equal to the predetermined reference value (step S1002: NO), the process proceeds to step S1006.

In step S1003, the management server 10 determines whether or not a body surface temperature T6 at the entry inspection site (touch point TP6) is lower than a predetermined reference value.

In this step, if the management server 10 determines that the body surface temperature T6 at the entry inspection site is lower than the predetermined reference value (step S1003: YES), the process proceeds to step S1004.

In contrast, if the management server 10 determines that the body surface temperature T6 at the entry inspection site is higher than or equal to the predetermined reference value (step S1003: NO), the process proceeds to step S1006.

Next, the management server 10 determines whether or not a body surface temperature T5 at the boarding gate (touch point TP5) is lower than a predetermined reference value (step S1004).

In this step, if the management server 10 determines that the body surface temperature T5 at the boarding gate is lower than the predetermined reference value (step S1004: YES), the process proceeds to step S1005.

In contrast, if the management server 10 determines that the body surface temperature T5 at the boarding gate is higher than or equal to the predetermined reference value (step S1004: NO), the process proceeds to step S1006.

In step S1005, the management server 10 determines whether or not the total number of touch points where a body surface temperature higher than or equal to the reference value is measured out of the touch points TP1 to TP7 is one or less.

In this step, if the management server 10 determines the total number of touch points defined as above is one or less (step S1005: YES), the process proceeds to step S1007. That is, the passenger is not considered as a person with a fever.

In contrast, if the management server 10 determines the total number of touch points defined as above is two or greater (step S1005: NO), the process proceeds to step S1006.

In step S1006, the management server 10 performs registration and update of the person-with-fever information DB 10d with person-with-fever information including the token ID of the passenger considered as a person with a fever, and the process proceeds to step S1007.

In step S1007, the management server 10 determines whether or not the determination process is completed for all the passengers.

In this step, if the management server 10 determines that the determination process is completed for all the passengers (step S1007: YES), the process ends.

In contrast, if the management server 10 determines that the determination process is not completed for all the passengers (step S1007: NO), the process returns to step S1001.

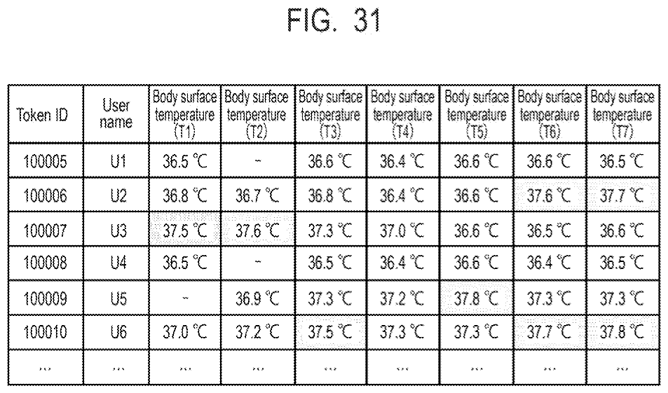

FIG. 31 is a diagram illustrating an example of measurement history information on body surface temperatures according to the present example embodiment. The body surface temperatures T1 to T7 are body surface temperatures measured at the check-in counter (touch point TP1), the baggage counter (touch point TP2), the security inspection site (touch point TP3), the departure inspection site (touch point TP4), the boarding gate (touch point TP5), the entry inspection site (touch point TP6), and the customs inspection site (touch point TP7), respectively.

For a user U1 having a token ID of “100005”, six values of the body surface temperatures T1 and T3 to T7 are acquired, and all the values thereof are lower than the reference value (37.5 degrees Celsius). In such a case, the management server 10 does not detect the user U1 as a person with a fever.

For a user U2 having a token ID of “100006”, seven values of the body surface temperatures T1 to T7 are acquired. In these values, two values of the body surface temperature T6 and the body surface temperature T7 are higher than the reference value (37.5 degrees Celsius). In such a way, even if all the body surface temperatures measured at the touch points TP1 to TP5 at the departure from the country are lower than the reference value, if the body surface temperature T5 at the touch point TP6 or TP7 at the entry to the country is higher or equal to the reference value (hereafter, referred to as “person-with-fever detection pattern A”), the management server 10 detects the user U2 as a person with a fever.

According to the person-with-fever detection pattern A, it is possible to detect a person with a fever with an emphasis on a body surface temperature in a procedure at entry to the country among a plurality of body surface temperatures at a plurality of touch points. The person-with-fever detection pattern A corresponds to the case where NO is determined in step S1002 or S1003 of FIG. 30 described above.

For a user U3 having a token ID of “100007”, seven values of the body surface temperatures T1 to T7 are acquired. In these values, two values of the body surface temperature T1 and the body surface temperature T2 are higher than or equal to the reference value (37.5 degrees Celsius). In such a way, if a body surface temperature higher than or equal to the reference value is recorded at two or more touch points (hereafter, referred to as “person-with-fever detection pattern B”), the management server 10 detects the user U3 as a person with a fever.

According to the person-with-fever detection pattern B, for example, even if the user U repeats a having-fever state and a non-having-fever state at the airport DA and the airport AA, it is possible to detect the user U as a person with a fever. The person-with-fever detection pattern B corresponds to the case where NO is determined in step S1005 of FIG. 30 described above.

For a user U4 having a token ID of “100008”, six values of the body surface temperatures T1 and T3 to T7 are acquired, and all the values thereof are lower than the reference value (37.5 degrees Celsius). In such a case, the management server 10 does not detect the user U4 as a person with a fever.

For a user U5 having a token ID of “100009”, six values of the body surface temperatures T2 to T7 are acquired. In these values, the value of the body surface temperature T5 is higher than the reference value (37.5 degrees Celsius). In such a way, even if all the body surface temperatures measured at touch points except for the boarding gate are lower than the reference value, if the body surface temperature T5 at a particular touch point TP5 (boarding gate) is higher than or equal to the reference value (hereafter, referred to as “person-with-fever detection pattern C”), the management server 10 detects the user U5 as a person with a fever.

According to the person-with-fever detection pattern C, it is possible to detect a person with a fever with an emphasis on a body surface temperature in a procedure immediately before boarding among a plurality of body surface temperatures at a plurality of touch points. The person-with-fever detection pattern C corresponds to the case where NO is determined in step S1004 of FIG. 30 described above.

For a user U6 having a token ID of “100010”, seven values of the body surface temperatures T1 to T7 are acquired. In these values, the body surface temperatures T3, T6, and T7 are higher than or equal to the reference value (37.5 degrees Celsius). In such a way, if the body surface temperature T3 at the touch point TP3 through which all the users U pass is higher than or equal to the reference value (hereafter, referred to as “person-with-fever detection pattern D”), the management server 10 detects the user U as a person with a fever.

According to the person-with-fever detection pattern D, it is possible to detect a person with a fever with an emphasis on a body surface temperature at a particular touch point among a plurality of body surface temperatures at a plurality of touch points. Note that the person-with-fever detection pattern is not limited to only the four person-with-fever detection patterns A to D described above and can be set as any pattern.

As described above, the present example embodiment can achieve an advantageous effect of being able to improve the accuracy in identifying a person with a fever in addition to the advantageous effect of the first example embodiment. Since the accuracy in identifying a contact person contacted with a person with a fever is also improved accordingly, it is possible to reliably guide the user U suspected of having contracted an infectious disease to an inspection lane. Further, since measurement history information on body surface temperatures at the first country and the second country have been acquired, it is possible to identify a time when a person with a fever started having the fever.

### Fourth Example Embodiment

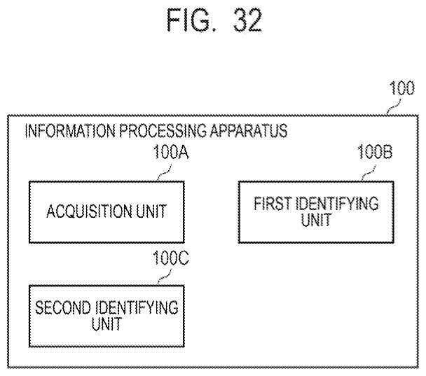

FIG. 32 is a function block diagram of an information processing apparatus 100 according to the present example embodiment. The information processing apparatus 100 includes an acquisition unit 100A, a first identifying unit 100B, and a second identifying unit 100C. The acquisition unit 100A acquires health information on users who boarded an airplane and seat information on the airplane. The first identifying unit 100B identifies a first person who has contracted an infectious disease out of users based on the health information. The second identifying unit 100C identifies a second person suspected of having the infectious disease infected from the first person in the airplane out of the users based on the seat information.

According to the present example embodiment, the information processing apparatus 100 that can efficiently detect a person suspected of having contracted an infectious disease is provided.

### Modified Example Embodiment

Although the present invention has been described above with reference to the example embodiments, the present invention is not limited to the example embodiments described above. Various modifications that can be understood by those skilled in the art can be made to the configuration and details of the present invention within the scope not departing from the spirit of the present invention. For example, it should be understood that an example embodiment in which a configuration of a part of any of the example embodiments is added to another example embodiment or an example embodiment in which a configuration of a part of any of the example embodiments is replaced with a configuration of a part of another example embodiment is also an example embodiment to which the present invention may be applied.

In the example embodiments described above, the configuration in which a positional relationship between a person with a fever and a contact person is defined in advance in the contact person definition information DB 10e of the management server 10 and this contact person definition information DB 10e is referenced to determine whether or not the user U is a contact person has been described. However, a method of identifying a contact person is not limited thereto. For example, a contact person contacted with a person with a fever may be identified based on a process result of a droplet scattering simulation inside an airplane performed by a supercomputer instead of being based on a database such as the contact person definition information DB 10e. On the contrary, the contact person definition information DB 10e may be defined based on a process result of a scattering simulation.

Further, a range of a contact person may be changed taking into consideration of not only the positional relationship with a person with a fever but also a time spent in an airplane. For example, the range of a contact person may be widened as the time spent is extended.

Further, although a case where a contact person in an airplane is identified has been described in the above example embodiments, a contact person outside the airplane may be identified and guided to an inspection lane. For example, with use of passage history information recorded in the passage history information DB 10b, it is possible to identify, as a contact person, the user U recognized to have stayed with a person with a fever for a certain period or longer or within a certain distance range at the same touch point. As an example, it is preferable to identify, as a contact person, a user U who was waiting in a line immediately in front of a person with a fever in the entry inspection site or the user U who stayed in the same space (for example, a lounge) as a person with a fever in an airport.

Further, although the case where a person with a fever is identified based on the body surface temperature of the user U measured at one or more touch points in an airport has been described in the above example embodiments, a method of identifying a person with a fever is not limited thereto. For example, data on a test result of a test for an infectious disease (for example, a genetic test, an antigenic test, and an antibody test) or data on a blood pressure, a blood oxygen level, a heart rate, or the like of the user U may be used as health information indicating the health condition of the user U. In such a case, even when the user U does not have a fever, the user U can be identified as a person having an infectious risk to others Further, a place where the test is performed is not limited to an inspection facility in an airport and may be an inspection facility of an outside medical institution. Furthermore, the number of types of health information used for identifying a person having an infectious risk is not limited to one, and multiple types of health information may be combined.

Further, although a person with a fever and a contact person contacted with the person with a fever are inspection targets in the example embodiments described above, all the passengers and crews in an airplane where the person with a fever was present may be identified as inspection targets. That is, the range of users U to be inspection targets can be changed to any range.

Furthermore, a contact person may be identified based on a group ID or information on a companion associated with a token ID. Specifically, since a user U for which the same group ID as a person with a fever is set in the token ID information DB 10a is a person who belongs to the same group and is expected to have many opportunities of contact with the person with a fever, the user U can be identified as a contact person.

Similarly, when information on a companion is associated with a token ID of a person with a fever in the operation information DB 10c, the companion can be identified as a contact person. When a group ID or information on a companion is taken into consideration, it is preferable that a companion can be identified as a contact person even when the companion was seated distant from the seat of the person with a fever.

The scope of each of the example embodiments also includes a processing method that stores, in a storage medium, a program that causes the configuration of each of the example embodiments to operate so as to implement the function of each of the example embodiments described above, reads the program stored in the storage medium as a code, and executes the program in a computer. That is, the scope of each of the example embodiments also includes a computer readable storage medium. Further, each of the example embodiments includes not only the storage medium in which the program described above is stored but also the individual program itself.

As the storage medium, for example, a floppy (registered trademark) disk, a hard disk, an optical disk, a magneto-optical disk, a CD-ROM, a magnetic tape, a nonvolatile memory card, or the like can be used. Further, the scope of each of the example embodiments also includes an example that operates on OS to perform a process in cooperation with another software or a function of an add-in board without being limited to an example that performs a process by an individual program stored in the storage medium.

The whole or part of the example embodiments disclosed above can be described as, but not limited to, the following supplementary notes.

### (Supplementary Note 1)

An information processing apparatus comprising:
an acquisition unit that acquires health information on users who boarded an airplane and seat information on the airplane;
a first identifying unit that identifies a first person out of the users based on the health information, the first person having contracted an infectious disease; and
a second identifying unit that identifies a second person out of the users based on the seat information, the second person being suspected of having the infectious disease infected from the first person in the airplane.

### (Supplementary Note 2)

The information processing apparatus according to supplementary note 1, wherein the second identifying unit identifies the second person based on a positional relationship with the first person in the airplane derived from the seat information.

### (Supplementary Note 3)

The information processing apparatus according to supplementary note 2, wherein the second identifying unit identifies the second person based on a definition table that defines the positional relationship in advance.

### (Supplementary Note 4)

The information processing apparatus according to any one of supplementary notes 1 to 3, wherein the second identifying unit identifies, as the second person, a user who used a seat located within a predetermined distance range from a seat of the first person.

### (Supplementary Note 5)

The information processing apparatus according to any one of supplementary notes 1 to 4, wherein the second identifying unit identifies, as the second person, a user who used a seat arranged in a predetermined direction from a seat of the first person.

### (Supplementary Note 6)

The information processing apparatus according to supplementary note 1, wherein the second identifying unit identifies, as the second person, a user who boarded the same airplane as the first person.

### (Supplementary Note 7)

The information processing apparatus according to any one of supplementary notes 1 to 6 further comprising:
a registration unit that registers user information to a database, the user information being associated with identification information on each of the users, a registered face image of each of the users, the health information on each of the users, flight information on the airplane, and the seat information on the airplane; and
a matching unit that performs face matching between a face image of each of the users captured in an airport and the registered face image,
wherein the acquisition unit acquires the user information from the database when the user is authenticated by the face matching.

### (Supplementary Note 8)

The information processing apparatus according to supplementary note 7 further comprising a guidance unit that guides a user identified as the first person or the second person to an inspection place in the airplane.

### (Supplementary Note 9)

The information processing apparatus according to supplementary note 8, wherein the guidance unit causes a display terminal that captures the face image in the airport to display guidance information including the inspection place.

### (Supplementary Note 10)

The information processing apparatus according to supplementary note 8 or 9,
wherein when the user is authenticated by the face matching, the acquisition unit further acquires procedure history information including a time and a procedure place where the user completed a predetermined procedure in the airport,
wherein based on the procedure history information, the second identifying unit identifies a third person suspected of having the infectious disease infected from the first person in the procedure place, and
wherein the guidance unit further guides a user identified as the third person to the inspection place.

### (Supplementary Note 11)

The information processing apparatus according to supplementary note 10, wherein the guidance unit outputs alert information to a communication terminal associated in advance with a user that is any of the first person, the second person, and the third person.

### (Supplementary Note 12]

The information processing apparatus according to any one of supplementary notes 7 to 11, wherein the health information is a body surface temperature of the user measured in the airport.

### (Supplementary Note 13)

The information processing apparatus according to any one of supplementary notes 1 to 11, wherein the health information is a test result of a test related to the infectious disease.

### (Supplementary Note 14)

The information processing apparatus according to supplementary note 12, wherein the first identifying unit identifies the first person based on the body surface temperature measured in the airport from which the airplane departed.

### (Supplementary Note 15)

The information processing apparatus according to supplementary note 12, wherein the first identifying unit identifies the first person based on the body surface temperature measured in the airport at which the airplane arrived.

### (Supplementary Note 16)

The information processing apparatus according to supplementary note 12, wherein the first identifying unit identifies the first person based on measurement history information including a plurality of body surface temperatures measured in the airport from which the airplane departed and the airport at which the airplane arrived, respectively.

### (Supplementary Note 17)

The information processing apparatus according to supplementary note 16, wherein based on a measurement time of the body surface temperatures included in the measurement history information, the first identifying unit further identifies a time when the first person started having a fever.

### (Supplementary Note 18)

An information processing method comprising: acquiring health information on users who boarded an airplane and seat information on the airplane;
identifying a first person out of the users based on the health information, the first person having contracted an infectious disease; and
identifying a second person out of the users based on the seat information, the second person being suspected of having the infectious disease infected from the first person in the airplane.

### (Supplementary Note 19)

A storage medium storing a program that causes a computer to perform:
acquiring health information on users who boarded an airplane and seat information on the airplane;
identifying a first person out of the users based on the health information, the first person having contracted an infectious disease; and identifying a second person out of the users based on the seat information, the second person being suspected of having the infectious disease infected from the first person in the airplane.

### REFERENCE SIGNS LIST

NW, NW1, NW2 network
10 management server
10a token ID information DB
10b passage history information DB
10c operation information DB
10d person-with-fever information DB
10e contact person information DB
20 check-in terminal
30 automatic baggage drop-off machine
40 security inspection apparatus
50, 70 automated gate apparatus
60 boarding gate apparatus
80 signage terminal
90 automatic customs gate apparatus
91 entry gate terminal
92 exit gate terminal
100 information processing apparatus
100A acquisition unit
100B first identifying unit
100C second identifying unit

What is claimed is:

1. An information processing apparatus comprising:
at least one memory storing instructions; and
at least one processor configured to execute the instructions to:
acquire health information on users who boarded an airplane and seat information on the airplane;
identify a first person out of the users based on the health information, the first person having contracted an infectious disease; and
identify a second person out of the users based on the seat information, the second person being suspected of having the infectious disease infected from the first person in the airplane.

2. The information processing apparatus according to claim 1, wherein the at least one processor identifies the second person based on a positional relationship with the first person in the airplane derived from the seat information.

3. The information processing apparatus according to claim 2, wherein the at least one processor identifies the second person based on a definition table that defines the positional relationship in advance.

4. The information processing apparatus according to claim 1, wherein the at least one processor identifies, as the second person, a user who used a seat located within a predetermined distance range from a seat of the first person.

5. The information processing apparatus according to claim 1, wherein the at least one processor identifies, as the second person, a user who used a seat arranged in a predetermined direction from a seat of the first person.

6. The information processing apparatus according to claim 1, wherein the at least one processor identifies, as the second person, a user who boarded the same airplane as the first person.

7. The information processing apparatus according to claim 1, wherein the at least one processor is further configured to execute the instructions to:
register user information to a database, the user information being associated with identification information on each of the users, a registered face image of each of the users, the health information on each of the users, flight information on the airplane, and the seat information on the airplane; and
perform face matching between a face image of each of the users captured in an airport and the registered face image,
wherein the at least one processor acquires the user information from the database when the user is authenticated by the face matching.

8. The information processing apparatus according to claim 7, wherein the at least one processor is further configured to execute the instructions to:
guide a user identified as the first person or the second person to an inspection place in the airplane.

9. The information processing apparatus according to claim 8, wherein the at least one processor causes a display terminal that captures the face image in the airport to display guidance information including the inspection place.

10. The information processing apparatus according to claim 8,
wherein when the user is authenticated by the face matching, the at least one processor further acquires procedure history information including a time and a procedure place where the user completed a predetermined procedure in the airport,
wherein based on the procedure history information, the at least one processor identifies a third person suspected of having the infectious disease infected from the first person in the procedure place, and
wherein the at least one processor further guides a user identified as the third person to the inspection place.

11. The information processing apparatus according to claim 10, wherein the at least one processor outputs alert information to a communication terminal associated in advance with a user that is any of the first person, the second person, and the third person.

12. The information processing apparatus according to claim 7, wherein the health information is a body surface temperature of the user measured in the airport.

13. The information processing apparatus according to claim 1, wherein the health information is a test result of a test related to the infectious disease.

14. The information processing apparatus according to claim 12, wherein the at least one processor identifies the first person based on the body surface temperature measured in the airport from which the airplane departed.

15. The information processing apparatus according to claim 12, wherein the at least one processor identifies the first person based on the body surface temperature measured in the airport at which the airplane arrived.

16. The information processing apparatus according to claim 12, wherein the at least one processor identifies the first person based on measurement history information including a plurality of body surface temperatures measured in the airport from which the airplane departed and the airport at which the airplane arrived, respectively.

17. The information processing apparatus according to claim 16, wherein based on a measurement time of the body surface temperatures included in the measurement history information, the at least one processor further identifies a time when the first person started having a fever.

18. An information processing method comprising:
acquiring health information on users who boarded an airplane and seat information on the airplane;
identifying a first person out of the users based on the health information, the first person having contracted an infectious disease; and
identifying a second person out of the users based on the seat information, the second person being suspected of having the infectious disease infected from the first person in the airplane.

19. A non-transitory storage medium storing a program that causes a computer to perform:
acquiring health information on users who boarded an airplane and seat information on the airplane;
identifying a first person out of the users based on the health information, the first person having contracted an infectious disease; and
identifying a second person out of the users based on the seat information, the second person being suspected of having the infectious disease infected from the first person in the airplane.
